(19) United States
(12) Patent Application Publication
(10) Pub. No.: US 2026/0227259 A1
(43) Pub. Date: Aug. 6, 2026

(54) PRESSURE SENSOR, FABRICATION AND USE THEREOF

(71) Applicant: NATIONAL UNIVERSITY OF SINGAPORE, Singapore (SG)

(72) Inventors: Chee Keong TEE, Singapore (SG); Wen CHENG, Singapore (SG); Xinyu WANG, Singapore (SG)

(73) Assignee: NATIONAL UNIVERSITY OF SINGAPORE, Singapore (SG)

(21) Appl. No.: 19/147,357

(22) PCT Filed: Jul. 12, 2024
(86) PCT No.: PCT/SG2024/050027

§ 371 Date: Jul. 11, 2025

(30) Foreign Application Priority Data
Jan. 13, 2023 (SG) 10202300109V

(51) Int. Cl. G01L 9/12; A61B 5/03

(52) U.S. Cl. CPC G01L 9/12; A61B 5/031; A61B 2503/40; A61B 2562/0247

(57) ABSTRACT

A pressure sensor structure, a method of fabricating a pressure sensor structure, a method of using the pressure sensor structure, and a device comprising the pressure sensor structure. The pressure sensor structure comprises a chamber having a closed bottom and an open top; an array of working electrode surfaces disposed within the chamber and extending from the bottom of the chamber towards the open top; and a counter electrode formed outside the chamber; wherein the chamber with the working electrode surfaces disposed therein is configured to entrap air when the chamber is immersed in a liquid; wherein the working electrode surfaces are electrically contactable from outside the chamber; and wherein, when the air is entrapped in the chamber, changes in a capacitance between the working electrode and the counter electrode are a measure for pressure changes in the liquid in which the chamber is immersed.

### FIELD OF INVENTION

The present invention relates broadly to a pressure sensor structure, a method of fabricating a pressure sensor structure, a method of using the pressure sensor structure, and a device comprising the pressure sensor structure.

### BACKGROUND

Any mention and/or discussion of prior art throughout the specification should not be considered, in any way, as an admission that this prior art is well known or forms part of common general knowledge in the field.

Existing pressure sensors typically rely on solid sensing elements. When applied in liquid environments, such pressure sensors experience a performance trilemma of linearity, hysteresis, and sensitivity.

Embodiments of the present invention seek to provide an alternative pressure sensor structure, a method of fabricating a pressure sensor structure, a method of using the pressure sensor structure, and a device comprising the pressure sensor structure, and which can preferably address at least one of the above problems.

### SUMMARY

In accordance with a first aspect of the present invention, there is provided a pressure sensor structure comprising:
a chamber having a closed bottom and an open top;
an array of working electrode surfaces disposed within the chamber and extending from the bottom of the chamber towards the open top; and
a counter electrode formed outside the chamber;
wherein the chamber with the working electrode surfaces disposed therein is configured to entrap air when the chamber is immersed in a liquid;
wherein the working electrode surfaces are electrically contactable from outside the chamber; and
wherein, when the air is entrapped in the chamber, changes in a capacitance between the working electrode and the counter electrode are a measure for pressure changes in the liquid in which the chamber is immersed.

In accordance with a second aspect of the present invention, there is provided a method of fabricating a pressure sensor structure comprising the steps of:
providing a chamber having a closed bottom and an open top;
disposing an array of working electrode surfaces within the chamber and extending from the bottom of the chamber towards the open top;
forming a counter electrode outside the chamber; and
configuring the chamber with the working electrode surfaces disposed therein is to entrap air when the chamber is immersed in a liquid;
wherein the working electrode surfaces are electrically contactable from outside the chamber; and
wherein, when the air is entrapped in the chamber, changes in a capacitance between the working electrode and the counter electrode are a measure for pressure changes in the liquid in which the chamber is immersed.

In accordance with a third aspect of the present invention, there is provided a method of using the pressure sensor structure of the first aspect to measure pressure, comprising the step of monitoring changes in the capacitance between the working electrode and the counter electrode as the measure for the pressure changes in the liquid in which the chamber is immersed.

In accordance with a fourth aspect of the present invention, there is provided a device comprising the pressure sensor structure of the first aspect.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1A shows a schematic fabrication flow or fabrication of a sensor according to an example embodiment.

FIG. 1B shows a scanning electron microscopy (SEM) image of a device structure with shadow mask cover, for a sensor according to an example embodiment.

FIG. 1C shows an SEM image of a half device structure with half shadow mask cover, for a sensor according to an example embodiment.

FIG. 1D shows an SEM image of a half device structure without shadow mask cover, for a sensor according to an example embodiment.

FIG. 1E shows a photo of a sensor according to an example embodiment.

FIG. 1F shows a photo of a sensor according to another example embodiment.

FIG. 2A shows a schematic illustration of a sensor according to an example embodiment. Inset: The equivalent circuit of the sensor.

FIG. 2B shows the loading-unloading performance of a sensor according to an example embodiment.

FIG. 2C shows a schematic illustration of the capacitor composition of a sensor according to an example embodiment, along with surfaces labelled I-IV with varying wetting properties used to tune the sensor's performance.

FIG. 2D shows a schematic illustration of the performance of a non-ideal pressure sensor with forward/backward threshold and an ideal pressure sensor with linear and hysteresis-free performance.

FIG. 2E shows a comparison of linearity of a sensor according to a preferred embodiment with reported liquid environment pressure sensors.

FIG. 2F shows an SEM image of a hexagon-array device without pillars used in for investigating the wetting properties of surfaces I-IV for use in example embodiments.

FIG. 2G shows an illustration of the composition of surface IV in a sensor according to an example embodiment, associated wetting properties and the resulting device performance.

FIG. 2H shows an illustration of the composition of surface III in a sensor according to an example embodiment, associated wetting properties and the resulting device performance.

FIG. 2I shows an illustration of the composition of surface II in a sensor according to an example embodiment, associated wetting properties and the resulting device performance.

FIG. 2J shows an illustration of the composition of surface I in a sensor according to a preferred embodiment, associated wetting properties and the resulting device performance.

FIG. 2K shows a graph illustrating the relationships between r.m.s. roughness and corresponding advancing angle and contact angle hysteresis of surfaces I-IV. Data are mean values±s.d. (five samples).

FIG. 2L shows a plot of advancing angles and forward thresholds of sensors according to example embodiments with surfaces I-IV. Data are mean values±s.d. (five samples).

FIG. 2M shows a plot of contact angle hysteresis and backward thresholds of sensors according to example embodiments with surface I-IV. Data are mean values±s.d. (five samples).

FIG. 2N shows a plot of contact angle hysteresis and hysteresis (%) of sensors according to example embodiments with surfaces I-IV. Data are mean values±s.d. (five samples).

FIG. 2O shows a graph illustrating the relationship between simulated contact area changes in hexagon-array devices (169 hexagonal chambers per device; side length, 40 μm) treated with surfaces I-IV, and measured capacitance changes of the corresponding devices, which are all linear (R²>0.99). Data are mean values±s.d. (five samples).

FIG. 3A shows SEM images of half a 3D-printed hexagon-walled pillar-array device (cross-section; scale bar, 200 μm), the dome-shaped pillar tip (scale bar, 10 μm) and the nanostructures ((120 s PAni)/Cr/Au/ODT SAMs, 400 nm thick in total) on the pillar surface (scale bar, 100 nm), for use in a sensor according to an example embodiment.

FIG. 3B shows confocal images of the top view and cross-sectional profiles of water-air interface movement in the hexagon-walled pillar-array structure for use in an example embodiment, under changing pressure (0-10 kPa). Scale bar, 200 μm,

FIG. 3C shows the loading-unloading behaviour in different pressure ranges, for a sensor according to an example embodiment.

FIG. 4A shows an SEM image of hexagon-walled pillar-array device structures with centre-to-centre spacings of the adjacent pillars 37.5 μm, according to an example embodiment.

FIG. 4B shows an SEM image of hexagon-walled pillar-array device structures centre-to-centre spacings of the adjacent pillars 50 μm, according to an example embodiment.

FIG. 4C shows an SEM image of hexagon-walled pillar-array device structures centre-to-centre spacings of the adjacent pillars 100 μm, according to an example embodiment.

FIG. 4D shows the sensitivity performance of sensors according to example embodiments, as a function of pillar spacing.

FIG. 4E shows the hysteresis performance of sensors according to example embodiments, as a function of pillar spacing.

FIG. 4F shows the linearity (R2) performance of sensors according to example embodiments, as a function of pillar spacing.

FIG. 5 shows a graph illustrating performance differences between devices with single hexagon, pillar array, and hexagon-walled pillar-array structures in the range of 0-15 kPa. All devices were treated with surface I.

FIG. 6 shows a comparison of cyclic tests of a sensor according to an embodiment with a commercial sensor (built-in resistance without external circuit) in the range of 0-50 Pa.

FIG. 7 shows the short-time Fourier transform spectrum of the sensor according to an example embodiment tested underwater with a sweep sound frequency 0-1 kHz applied.

FIG. 8 shows a graph of 1,000 cycles of durability test of the sensor according to an example embodiment in the range of 0-5 kPa.

FIG. 9 shows a graph illustrating advancing angle and contact angle hysteresis comparison of ultra-slippery surface (Surface I with 5 cSt silicone oil) before and after 10,000 times dipping in 1×PBS solution. Data are represented as mean values±s.d. (3 samples) with the corresponding data points overlapped.

FIG. 10A shows a graph illustrating the linearity (R² was adopted for evaluation) variation over time of a sensor immersed under 1×PBS solution with maintained pressures at 0 kPa, 10 kPa, and 15 kPa (3 devices at each pressure) and the performance restored by reapplying the lubricant. Data are represented as mean values±s.d. (3 samples).

FIG. 10B shows a graph illustrating the hysteresis variation over time of a sensor immersed under 1×PBS solution with maintained pressures at 0 kPa, 10 kPa, and 15 kPa (3 devices at each pressure) and the performance restored by reapplying the lubricant. Data are represented as mean values±s.d. (3 samples).

FIG. 10C shows a graph illustrating the sensitivity variation over time of a sensor immersed under 1×PBS solution with maintained pressures at 0 kPa, 10 kPa, and 15 kPa (3 devices at each pressure) and the performance restored by reapplying the lubricant. Data are represented as mean values±s.d. (3 samples).

FIG. 11 shows (left) a photograph of a sensor according to an example embodiment under turbulence (up to a Reynolds number of ~6,400); (right) a graph illustrating a stability test of the sensor according to an example embodiment under turbulence with different Reynolds numbers (0-6,400-0) generated by different stir speeds (0-1,000-0 r.p.m.) under simultaneous cyclic pressure (0-10 kPa).

FIG. 12 shows a graph and schematic drawing illustrating the capacitance response of a sensor according to an example embodiment tested at different facing orientations against gravity.

FIG. 13 shows a comparison of sensing performance of a sensor according to an example embodiment exposed to bovine serum albumin (BSA) solution, and with encapsulation according to another example embodiment, respectively.

FIG. 14 shows a photo of a sensor encapsulated with Ecoflex/Parylene dome with 1×PBS solution filled inside, according to an example embodiment.

FIG. 15A shows a graph illustrating the performance of a sensor before and after encapsulation, according to example embodiments.

FIG. 15B shows a graph illustrating cyclic test of a sensor according to example embodiment in the range of 0-10 kPa before and after encapsulation.

FIG. 16 shows the capacitance response of a sensor according to an example embodiment under different temperatures. The devices were tested in 1×PBS with cyclic pressure applied.

FIG. 17A shows a photograph of a rat cranium mounted with a sensor according to an example embodiment and a high-resolution barometer.

FIG. 17B shows a schematic illustration of the working principle of wireless ICP sensing, according to an example embodiment.

FIG. 17C shows frequency readings as a function of pressure. Inset: photograph of a sensor according to an example embodiment.

FIG. 18 shows in vivo wireless monitoring of ICP in a rat by a sensor according to an example embodiment (bottom). The applied pressure was tracked by a commercial barometer (top).

FIG. 19 shows a schematic illustration of the working mechanism of a surgical grasper equipped with a sensor according to an example embodiment for pressure sensing during surgery. One of the grasper jaws was mounted with compressible liquid chambers, which are connected to sensor chambers by PBS-filled thin tubes. As the grasper grasps the tissue, the pressure is transmitted to the sensors according to example embodiments through the squeezed liquid.

FIG. 20A shows a technical drawing illustrating the dimensions of the receptors in the grasper of FIG. 19. Elastic cap with protrusions (top). Rigid liquid chamber (bottom). The unit used is millimeters.

FIG. 20B shows a photograph of a grasper integrated with receptor chambers on the jaw. According to an example embodiment.

FIG. 20C shows a photograph of sensors according to example embodiments in sealed chambers, for a grasper according to an example embodiment.

FIG. 20D shows the sensing performance of sensors according to example embodiments integrated with tubing chambers. The sensors exhibit high linearity (R²=0.99646±0.00112), low hysteresis (5.66±1.38%) and high sensitivity (215.2±5.6 pF N⁻¹) (three devices).

FIG. 20E shows graphs illustrating values of grasping forces recorded over time by the sensors according to example embodiments integrated with the grasper while grasping an artificial tissue. Several scenarios were performed, including grasp adaptation with position sensing, detection of insufficient-force-induced slippage, and firmly grasping with appropriate forces. Different shading regions denote the operation periods.

FIG. 21 shows a photograph of the test setup for pressure sensing performance characterization of sensors according to example embodiments.

FIG. 22A shows a graph illustrating the advancing angle variations of surfaces coated with 350 cSt and 5 cSt silicone oil over time, according to example embodiments. Data are represented as mean values±s.d. (3 samples).

FIG. 22B shows a graph illustrating the contact angle hysteresis variations of surfaces coated with 350 cSt and 5 cSt silicone oil over time, according to example embodiments. Data are represented as mean values±s.d. (3 samples).

FIG. 23A shows a graph illustrating the linearity (R² was adopted for evaluation) variations of sensors coated with 350 cSt and 5 cSt silicone oil over time, according to example embodiments. The devices were immersed in 1×PBS solution and subjected to constant pressures of 15 kPa. The sensing performance was restored by reapplying corresponding silicone oil. Data are represented as mean values±s.d. (3 samples).

FIG. 23B shows a graph illustrating the hysteresis variations of sensors coated with 350 cSt and 5 cSt silicone oil over time, according to example embodiments. The devices were immersed in 1×PBS solution and subjected to constant pressures of 15 kPa. The sensing performance was restored by reapplying corresponding silicone oil. Data are represented as mean values±s.d. (3 samples).

FIG. 23C shows a graph illustrating the sensitivity variations of sensors coated with 350 cSt and 5 cSt silicone oil over time, according to example embodiments. The sensors were immersed in 1×PBS solution and subjected to constant pressures of 15 kPa. The sensing performance was restored by reapplying corresponding silicone oil. Data are represented as mean values±s.d. (3 samples).

FIG. 23D shows a graph illustrating the performance at the initial state, after 5 days, and after reapplying oil for a 5 cSt coated sensor according to an example embodiment.

FIG. 23E shows a graph illustrating the performance at the initial state, after 5 days, and after reapplying oil for a 350 cSt coated sensor according to an example embodiment.

FIG. 23F shows a graph illustrating the effect of viscosity of coated oil on hysteresis performance of sensors according to example embodiments coated with the same volume of oils (5 μL, 0.1% v/v silicone oil diluted in n-heptane) and tested at the same pressure (0-10 kPa). Data are represented as mean values±s.d. (3 samples) with corresponding data points overlapped.

FIG. 24 shows a graph illustrating the sensing performance of a sensor coated with 1k cSt silicone oil subjected to different pressure change rates, according to example embodiments.

FIG. 25A shows a graph illustrating the effect of pressure changing rate on sensitivity of sensors according to example embodiments coated with silicone oil (5 cSt). Data are represented as mean values±s.d. (3 samples).

FIG. 25B shows a graph illustrating the effect of pressure changing rate on linearity of sensors according to example embodiments coated with silicone oil (5 cSt). Data are represented as mean values±s.d. (3 samples).

FIG. 25C shows a graph illustrating the effect of pressure changing rate on hysteresis of sensors according to example embodiments coated with silicone oil (5 cSt). Data are represented as mean values±s.d. (3 samples).

FIG. 26A shows a graph illustrating the effect of test signal frequency on a sensor according to an example embodiment performance, in a sweep from 20 Hz to 2 MHz without external pressure applied.

FIG. 26B shows a graph illustrating the capacitance response at different frequencies with cyclic pressure applied for a sensor according to an example embodiment.

FIG. 27A shows a graph illustrating the effect of volume of coated diluted oil on sensitivity of a sensor according to example embodiment, with the volume of coated diluted oil (0.10% v/v of 5 cSt silicone oil diluted in n-heptane). Data are represented as mean values±s.d. (5 samples).

FIG. 27B shows a graph illustrating the effect of volume of coated diluted oil on linearity of a sensor according to example embodiment with the volume of coated diluted oil (0.10% v/v of 5 cSt silicone oil diluted in n-heptane). Data are represented as mean values±s.d. (5 samples).

FIG. 27C shows a graph illustrating the effect of volume of coated diluted oil on hysteresis of a sensor according to example embodiment with the volume of coated diluted oil (0.10% v/v of 5 cSt silicone oil diluted in n-heptane). Data are represented as mean values±s.d. (5 samples).

FIG. 28A shows photos of air bubbles on a sensor according to an example embodiment under a series of negative pressures.

FIG. 28B shows a graph illustrating the sensing performance of a sensor according to an example embodiment within the pressure range of −2 kPa to 15 kPa.

FIG. 28C shows a graph illustrating the sensing performance of a sensor according to an example embodiment within the pressure range of −4 kPa to 15 kPa.

FIG. 28D shows a graph illustrating the sensing performance of a sensor according to an example embodiment within the pressure range of 0 to 50 kPa.

FIG. 29 shows a flowchart illustrating a method of fabricating a pressure sensor structure, according to an example embodiment.

### DETAILED DESCRIPTION

Embodiments of the present invention provide a miniature pressure sensor that enables accurate static and dynamic pressure sensing in liquid environments or with liquid as operating medium, with high sensitivity, ultrahigh linearity, and ultralow hysteresis. The sensor according to example embodiments employs the aero-elasticity, a delicate structural design, a multiscale ultra-slippery surface, and the electrical double layer effect at the electrode-liquid interface to capacitively output the change of liquid pressure. The sensor according to an example embodiment can be provided in a miniature size and can assess pressure changes in turbulence, in vivo, and under acoustic vibrations. It is noted that example embodiments are not limited to pressure sensors intended for “direct” operation in liquid environments, but also include pressure sensors with liquid as the operating medium intended for use in various environments, including in non-liquid environments.

Preferred embodiments of the present invention use a mechanism of pressure-modulated solid-liquid-liquid-gas four-phase line movement in a walled pillar array structure with a surface that has low surface energy, is hydrophobic, and is frictionless in terms of sliding friction of the relevant liquid, e.g. water, together also referred to as ultra-slippery surface herein.

Embodiments of the present invention are bioinspired from the surface of the lotus leaf and nepenthes (pitcher plant). The surface of lotus leaf is superhydrophobic with low surface energy hierarchical microstructures. The surface can trap an air layer when a lotus leaf is submerged in water, which can be observed as a bright reflective layer. The trapped air layer is elastic and can deform when water pressure changes. Inspired by this phenomenon, a pressure sensor that uses the aero-elasticity of entrapped air and multiscale microstructures treated as ultra-slippery is provided according to example embodiments. In the example embodiments, the sensor comprises a micro-structured electrode and a flat counter electrode. The micro-structured electrode according to example embodiments is a pillar array with dome-shaped tips and surrounded with a wall, for example, but not limited to, a hexagonal wall in the example embodiments described herein. In an example embodiment of a fabrication method, the structure is printed using a two-photo polymerization tool. In the example embodiment, the surface of the micro-structured electrode is deposited with gold first to make it conductive, then electrochemically deposited with polyaniline nanowires to give the surface nanotextures, then another layer of gold is deposited on the polyaniline nanowires to enhance the conductivity of the nanotexture layer and provide an interface for oleophilic treatment, then the gold surface is treated with 1-Octadecanethiol to make it oleophilic, finally the surface is coated with silicone oil to render it ultra-slippery.

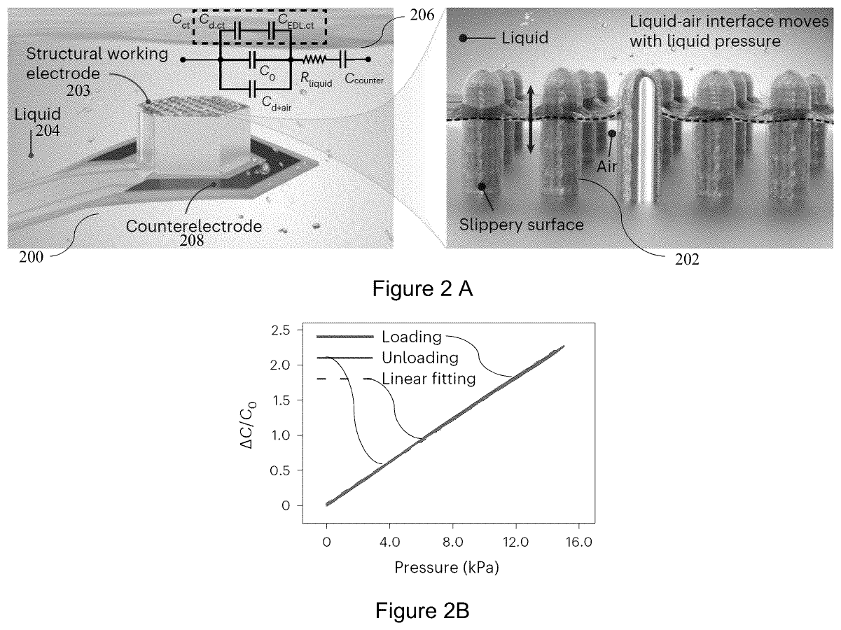

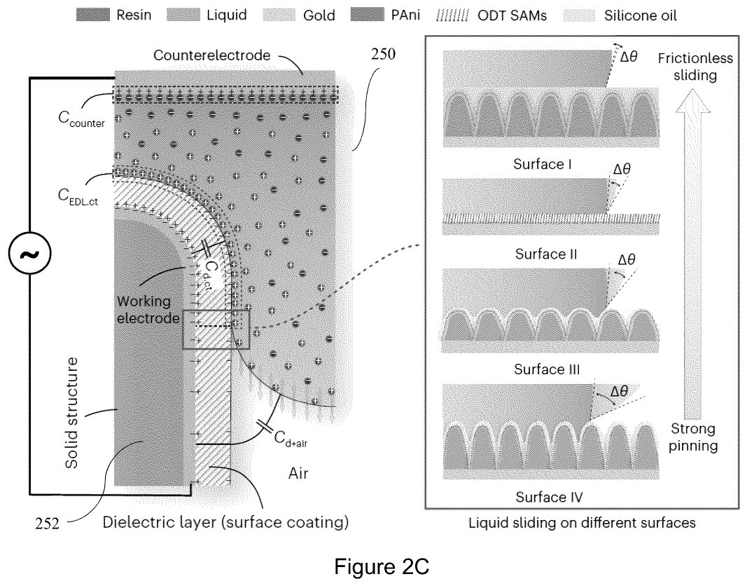

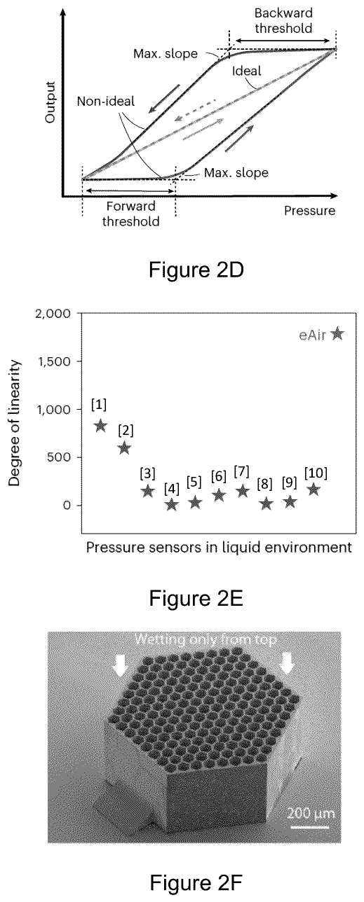

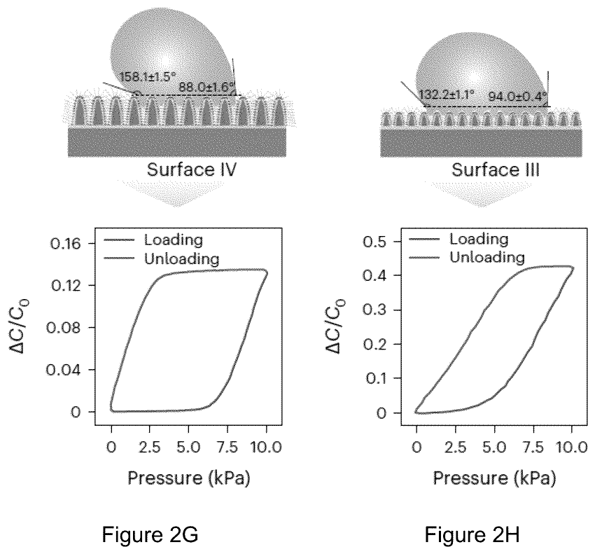

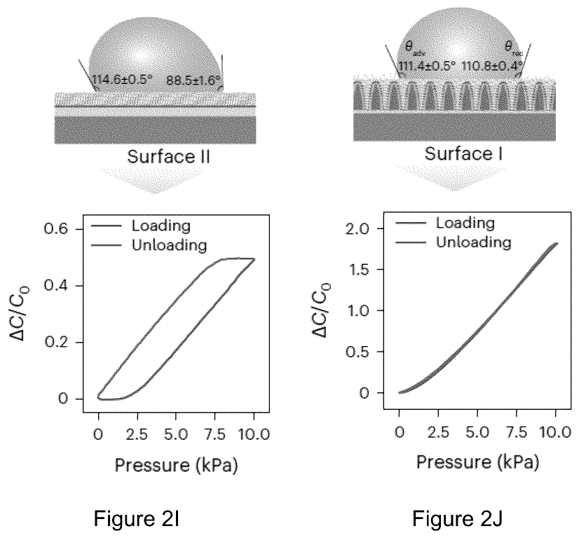

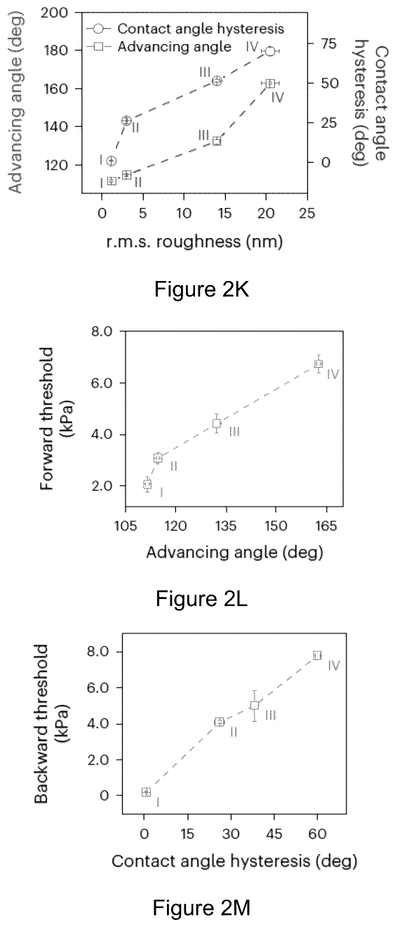

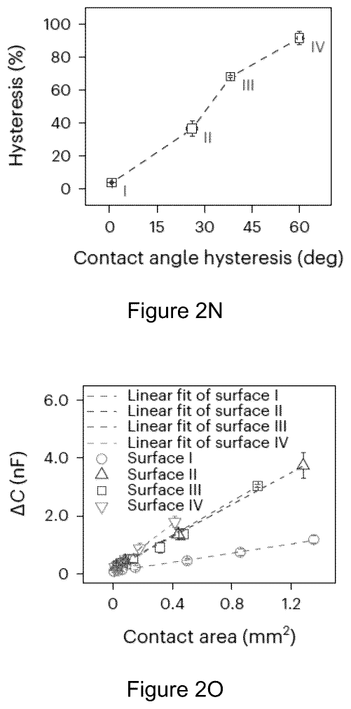

The sensor according to example embodiments is pressure-sensitive when submerged or encased in a liquid which contains ions that can move in the presence of an electric field so that an electrical double layer capacitor can be formed at the electrode-liquid interface, such as salt water. The sensor transduces pressure changes to capacitance changes. When the liquid pressure changes, the liquid will wet or dewet the surface of the microstructured electrode and change the contact area between the liquid and the electrode, and thus the interfacial capacitance response to the pressure changes. The sensor outputs the change by measuring the capacitance between the counter electrode and the microstructured electrode. The capacitance can be tested with an LCR meter or a capacitance meter under changing liquid pressure. In the example embodiment, the designed structure and low surface energy property helps trap air in the sensor chamber. As will be appreciated by a person skilled in the art, surface energy is related to the wetting properties of the surface. Materials with higher surface energy are easier to wet out than materials with lower surface energy, which means that e.g. water has poor wettability on low surface energy surfaces, and hence has a larger contact angle. When the microstructured electrode has a low surface energy, the water will not be completely wetted in the chamber, thus ensuring that the chamber has air trapped inside the chamber, for the formation of the layer of air that can be compressed (refer also FIG. 2A described below).

The conductive surface underneath the coated oil and the electrical double layer effect at the oil-liquid interface enable large capacitance outputs. The elasticity of the entrapped air enables the reversible wetting and dewetting process when liquid pressure changes. The surface of the device according to example embodiments mimics the surface of nepenthes and gives a low-friction, preferably frictionless, surface, also referred to as ultra-slippery herein, to preferably enable a hysteresis-free performance. The hexagon-walled pillar-array structure in example embodiments described herein enables the linear relationship between the pressure and output capacitance.

The working mechanism of example embodiments includes: 1. air elasticity; 2. walled pillar-array structure design; 3. ultra-slippery surface, i.e. a surface that has low surface energy, is hydrophobic, and is frictionless in terms of sliding friction of the liquid; 4. interfacial capacitance. The structure of the sensor according to example embodiment creates a space for entrapping air, and the liquid does not fully wet into the structure's space (also referred to as chamber herein). Because air is elastic and compressible, the liquid (e.g. 1×PBS solution during testing) can enter and exit the chamber with liquid pressure. The interfacial capacitance, which is made up of components of the electric double layer capacitances and surface coating dielectric layer capacitances, is proportional to the contact area between the 3D electrode surface and the liquid. The ultra-slippery surface preferably allows for drag and friction-free motion of the solid-liquid-liquid-air interface at the surface of the 3D electrodes. The structure design according to example embodiments preferably forces the liquid's wetting direction to be along the pillars, which, combined with air elasticity, allows the contact area between the liquid and solid to be linearly proportional to the liquid pressure.

In operation, the liquid-air interface sags towards the inner chamber of the sensor according to example embodiments when an external liquid pressure is applied. At the initial state, with small external pressure (0-~2 kPa) being applied, only the center of the liquid-air interface sags and the center pillars pierce the interface, increasing the contact area between the pillars and the liquid, and hence capacitance, allowing the sensor to respond to small pressures. The higher the pressure, the deeper the pillars pierce into the liquid from center to side/periphery of the chamber, and the contact area grows linearly with pressure. As the pressure rises (>~2 kPa & <15 kPa), the entire liquid-air interface moves synchronously into the chamber, increasing the contact area linearly with the pressure. When the pressure exceeds 15 kPa, the air becomes harder to compress due to the air compressing property. The air pressure in the chamber is inversely proportional to the air volume, thus the contact area cannot maintain the linear relationship with the external liquid pressure. Because of the electrode surface's ultra-slippery surface property in example embodiments, when the external pressure decreases, the liquid-air interface returns to the initial state with a linear relationship to the pressure and preferably without hysteresis effect.

It is noted that adhesion between liquid molecules and a nanoscopic rough solid surface can cause pinning of the liquid. This pinning effect can cause the motion of contact lines to lag which would contribute to poor responses of contact line motion to pressure changes. Hence, in an example embodiment, an ultra-slippery surface is provided on the pillars to reduce, and preferably eliminate, pinning of the liquid and associated lag of motion of the contact lines.

The bioinspired aero-elastic pressure sensor according to example embodiments differs from conventional solid-state pressure sensors and can exhibit near-ideal sensing performance. The strategy of using multi-scale structural electrodes for air trapping and the elimination of surface pinning at nanoscale creates a membrane-free pressure sensor design according to example embodiments, which can exhibit near ideal performance in preferred embodiments. For a preferred embodiment described herein, the highest linearity to-date that simultaneously possess ultra-low hysteresis and high sensitivity in liquid environments was achieved (ultra-high linearity (R-square=0.99944±0.00015, non-linearity=(1.49±0.17)%), ultra-low hysteresis ((1.34±0.20)%), and high sensitivity (79.1±4.3 pF/kPa)). The design rules for such membrane-free sensors according to example embodiments are further elucidated below. A wireless and implantable device to monitor intracranial pressure, and a sensorized surgical grasper to feedback grasping force during laparoscopic surgeries were demonstrated as example industrial application cases. The flexibility of structural design and surface modification can inspire further sensing performance tuning and customization for various application scenarios according to various example embodiments, such as pressure monitoring in dynamic fluids, or as acoustic underwater hydrophones. It is anticipated that the proposed strategy for pressure sensing in diverse liquids and ambient pressures according to example embodiments can be broadly applicable to pressure monitoring applications with ultra-precision and sensitivity.

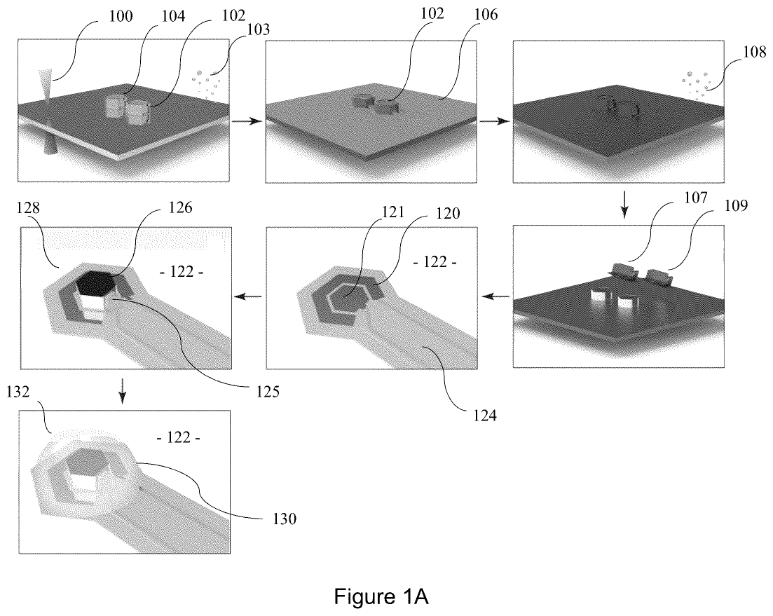

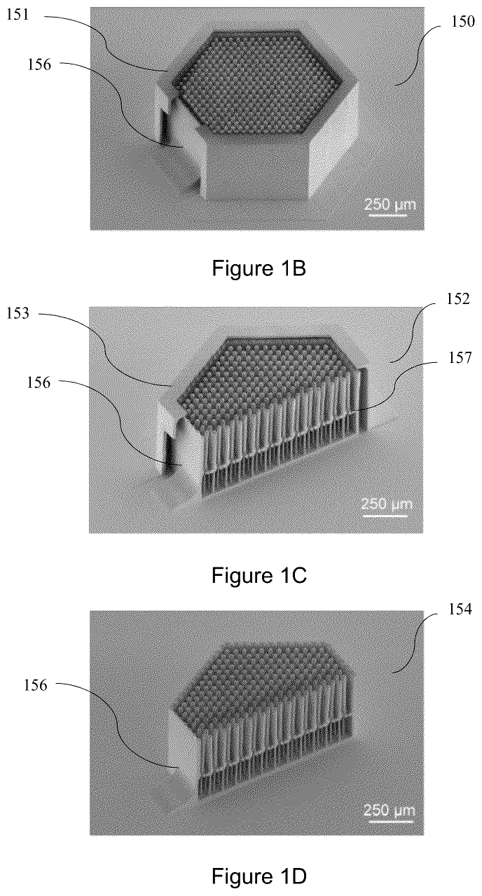

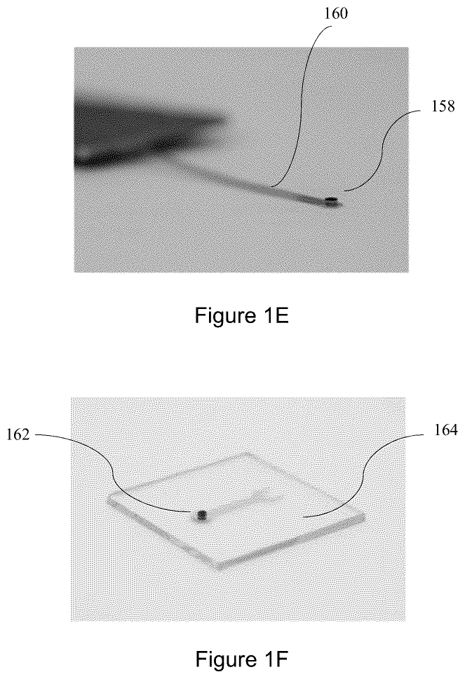

FIG. 1A shows the fabrication flow and FIGS. 1B-F show images of the sensor according to an example embodiment, specifically FIG. 1B shows an SEM image of a device structure 150 with shadow mask cover 151 for a sensor according to an example embodiment, FIG. 1C the half device structure 152 with shadow mask 153 cover, and FIG. 1D the half device structure 154 without shadow mask cover. The mask cover 151, 153 was designed to reduce the exposed area on the outside of devices during sputtering and to form the pattern of electrode 156 for wiring. The mask cover 151, 153 was removed after surface treatment. FIG. 1E shows a photo of a device 158 mounted on flexible polyimide substrate 160. FIG. 1F shows a photo of a device 162 mounted on glass substrate 164.

With reference to FIG. 1A, the detailed fabrication steps according to an example embodiment are:

### 1. Structure Design, Printing and Sputtering:

The structures of devices were designed in SolidWorks and printed using a two-photon polymerization lithography system (Photonic Professional GT2, Nanoscribe GmbH & Co.KG), indicated at numeral 100. For the hexagon-walled pillar-array structure e.g. 102 according to an example embodiment, the outermost length of each side of the hexagon wall is 615 μm, the height is 520 μm, and the wall thickness is 20 μm. The periodic pillars were in hexagonal lattice alignment with distance of 50 μm. The diameter and height of pillars with dome tips are 25 μm and 500 μm, respectively. The lower part of the pillars was reduced to 12.5 μm to expand the volume of air contained within the chamber. A mesh is used towards the bottom of the pillars for additional securing of the pillars (see e.g. numeral 157 in FIG. 1C). Covers were added outside the structures as shadow masks for sputtering (compare also FIGS. 1B-D), thus patterns of electrodes (see numeral 156 in e.g. FIG. 1B) were formed for wiring, and the initial exposed working electrode area were restrained. A negative photoresist IP-S (Nanoscribe GmbH & Co.KG) and a 25× objective lens were used for printing. The printed structures were developed in propylene glycol monomethyl ether acetate (PGMEA, Sigma-Aldrich) for 50 min. Then the samples were washed in isopropanol (Fisher Scientific) and Novec™ 7100 Engineered Fluid (Sigma-Aldrich) for 5 min respectively before drying. The device structures were then sputtering-coated with 50 nm/600 nm Cr/Au, indicated at numeral 103, to make the 3D structure conductive. It is noted that the process can be suitable for mass-production of 3D structures for the pressure sensors, as illustrated by the fabrication of multiple 3D structures 102, 104 in one printing process.

### 2. Surface Treatment:

Different surface treatments were applied in various example embodiments, also referred to a surfaces I-VI herein. The surface treatment according to a preferred example embodiment, also referred to as “surface I” herein, was achieved by depositing polyaniline (PAni) nanowires, indicated at numeral 106, followed by sputtering Cr/Au layer, 1-Octadecanethiol treatment, and oil coating, together indicated at numeral 108.

2.1 PAni nanowires deposition to create a porous rough nanotexture on device microstructure. The growth of PAni nanowires was done by electrochemical deposition in an example embodiment. A solution of 2 M sulfuric acid (95-97%, Sigma-Aldrich) and 0.5 M distilled aniline (Sigma-Aldrich) were prepared. Then the device e.g. 102 was immersed in the prepared solution and applied a vacuum to force the liquid into the 3D structures. For the electrochemical deposition process, an electrochemical cell is assembled in a three-electrode configuration in which the counter electrode is platinum (Pt), the reference electrode is silver/silver chloride, and the working electrode is the metalized 3D-printed structures. Then the deposition was conducted with an electrochemical workstation (Zahner Zennium E) at a constant potential of 0.75 V for 120 s. The device was washed in deionized water and dried in an oven at 70° C. for 1 hour.

2.2 Sputtering Cr/Au. The PAni nanowires surface was sputtered with another layer of Cr/Au to create an interface for hydrophobic treatment. The thicknesses of Cr and Au are 10 nm and 120 nm respectively.

2.3 1-Octadecanethiol (ODT) treatment. The ODT treatment was done to make the surface of gold coated PAni nanowires hydrophobic and oleophilic. The treatment was done by preparing the solution of 0.5% v/v ODT/absolute ethanol and immersing the devices in overnight. The devices were taken out, washed with ethanol, and dried with an air gun.

2.4 Oil coating. The oil coating is to lubricate the surface to be ultra-slippery. The diluted 0.10% silicone oil (5 cSt, Sigma-Aldrich) in n-heptane (anhydrous, 99%, Sigma-Aldrich) was applied onto the surface after ODT treatment. The silicone oil was drop-casted on the structural working electrode with control of volume by using a microsyringe to achieve ultra-slippery performance. The diluted silicone oil fills the voids in the porous nanotexture. The solvent (n-heptane) was evaporated naturally at room temperature. Note that the oil coating step was done after the assembly of the devices.

### 3. Counter Electrode Fabrication:

Flat counter electrode 120 and an electrode 121 for connecting the 3D printed structure 126 were fabricated on glass slides 122 by lithography. Generally, 10 nm chromium and 70 nm gold were sputtered on cleaned glass slides (a flexible polyimide substrate in different example embodiments), then a layer of spin-coated positive photoresist (AZ1512 HS, Merck) was patterned on the glass slide surface using ultraviolet lithography, and then etched with gold etchants (Sigma-Aldrich) and chromium etchants (22% ammonium cerium(IV) nitrate (Sigma-Aldrich) and 8% acetic acid (TCI) in deionized water), respectively, to obtain the electrode patterns 120, 121. The polyimide insulation layer 124 was prepared by a positive photoresist-assisted lithography method. Consequently, a 15.0-16.0% poly(pyromellitic dianhydride-co-4,4′-oxydianiline) amic acid solution (Sigma-Aldrich) was spin coated onto the substrate. A soft bake step was applied at 100° C. for 3 min, followed by spin coating with AZ1512 HS with a subsequent soft bake at 100° C. for 1 min. The samples were exposed to ultraviolet, followed by development in 2.38% tetramethylammonium. The alkaline tetramethylammonium developer continuously etched the uncrosslinked amic acid layer after washing the ultraviolet-exposed regions of the AZ1512 HS layer. Samples were rinsed in deionized water, and the AZ photoresist was removed with propylene glycol monomethyl ether acetate. After that, a hard bake was performed at 200° C. for 2 h. For the flexible flat electrode fabrication, additional initial steps of amic acid solution spin coating and lithography were added to make a flexible substrate, and then followed by the same steps mentioned above.

### 4. Device Assembly:

The structural working electrodes, i.e. the metalized 3D-printed structures e.g. 104, with different types of surface treatments were manually transferred and electrically connected to the flat electrode 121 on the glass slides (or a flexible polyimide substrate in different example embodiments) with adhesion of silver paste (Paron-910, Chang Sung Corp.). It is noted that the printed masks 107, 109 were removed after the surface treatment. The exposed conductive wiring area 125 outside the 3D printed structure 126 was encapsulated with SU-8 2002 (Micro Chem). Depending on the intended application environment of the pressure sensor in various example embodiments, the sensor may be used as indicated at numeral 128. In another example embodiment, the 3D printed structure e.g. 126 was filled with 1× phosphate buffered saline (PBS, BioWhittaker) and then encapsulated in an Ecoflex/Parylene-C pre-shaped dome 130 while being submerged in PBS to provide a pressure sensor 132 intended for various environments using liquid, in this example PBS, as operating medium.

Operation mechanism according to example embodiments.

FIG. 2A shows a schematic illustration of a sensor 200 according to an example embodiment. The surfaces of pillars e.g. 202 serve as the working electrode 203 and are modified to be ultra-slippery by infusing a lubricant into the conductive nanostructures, as described with reference to step “2. Surface treatment” above. The liquid 204 can wet/dewet the pillar electrode 203 under varying liquid pressures, preferably without contact angle hysteresis. This process changes the liquid 204—working electrode 203 contact area, and therefore the capacitance.

The inset shows the equivalent circuit 206 of the sensor 200, where C_{counter}, is the electrical double layer (EDL) capacitance at the liquid 204—counter electrode 208 interface; C_{EDL,ct}, is the EDL capacitance at the liquid 204—the surface coating of pillars e.g. 202 interface; C_{d,ct}, the dielectric capacitance between the working electrode 203 and the surface coating of the pillars e.g. 202—liquid 200 interface; C_{d+air}, is the capacitance between the working electrode 203 and the liquid 200 through the trapped air; C₀ is the interfacial capacitance of the non-active area outside the 3D structure.

FIG. 2B shows the pressure-sensing response of a sensor according to an example embodiment.

FIG. 2C shows a schematic illustration of the capacitor composition of a sensor 250 according to example embodiments, along with schematic illustrations of pillar e.g. 252 surfaces labelled I-IV with varying wetting properties used to tune the sensor's 250 performance, where the circuit labels correspond to the inset in FIG. 2A, and Δθ is the contact angle hysteresis of the surfaces. As surface roughness decreases from IV to I, Δθ also decreases, indicating a transition from strong pinning to frictionless sliding of liquid. These changes in surface properties impact sensor performance in various example embodiments.

FIG. 2D shows a schematic graph of the performance of a non-ideal pressure sensor with forward/backward threshold and an ideal pressure sensor with linear and hysteresis-free performance.

FIG. 2E shows a comparison of linearity of a sensor according to an example embodiment, labelled eAir, with reported liquid environment pressure sensors, see refs. [1]-[10]. To highlight the differences, the degree of linearity (1/(1−R²)) is defined for comparison. R² (coefficient of determination) is the value of the linear fitting results of the performance curves of corresponding devices: the higher the degree of linearity, the better the linearity.

Optimisation of sensors according to example embodiments.

To utilize the entrapped air for electronic pressure sensing, the contact line motion is preferably modulated to transduce pressure to capacitance using the EDL. Shifting contact lines change the contact area of the solid-liquid interface and thus change the interfacial capacitance created by the EDL. The correlation between the advancing angle and sensing thresholds was revealed and investigated according to example embodiments, as well as the correlation between contact angle hysteresis and sensing hysteresis. These results show the potential for optimizing pressure sensing by exquisite control of the structural design and surface chemical properties according to example embodiments. The sensing approach according to example embodiments is radically different from conventional solid-state pressure sensors. Such conventional pressure sensors are either inapplicable or have performance limitations in liquid environments, exhibiting, for example, low linearity, high hysteresis and low sensitivity. Thus, these conventional sensors are less suitable for aqueous environments or biomedical applications within body cavities, or for sensors using a liquid working medium in various environments.

Contrary to these solid-state pressure sensors, the sensing strategy according to a preferred embodiment of the present invention utilizes the substantially pinning-free contact line motion of a multiscale structured solid-liquid-liquid-gas multiphasic system to create a new type of aero-elastic capacitive pressure sensor, also termed eAir™ sensor herein. The eAir sensor according to example embodiments were provided as miniature (~0.5 mm³) sensors using a hexagon-walled pillar-array microstructure employing ultra-slippery surfaces inspired by the Nepenthes pitcher plant. The sensors according to example embodiments can measure subtle pressure fluctuations in liquid with ultralow hysteresis (1.34±0.20%), high sensitivity (79.1±4.3 pF kPa⁻¹) and extremely high linearity (R²=0.99944±0.00015; nonlinearity, 1.49±0.17%) compared with other pressure sensors operating in liquid environments, as shown in FIG. 2E. As described in detail below, it was demonstrated that eAir sensors according to example embodiments can operate under various complex conditions including turbulent flow, in in vivo biological environments and during laparoscopic procedures for ultrasensitive pressure monitoring.

It is noted that for regular open-cell arrays of microscale pillars fabricated using two-photon polymerization three-dimensional printing nanolithography and manufactured into pressure-sensitive devices by metallizing and hydrophobilizing, the surface exhibited poor repeatability, along with obvious hysteresis (21.8±7.7%), and forward (1.6±0.5 kPa) and backward thresholds (2.2±0.3 kPa). It was recognized by the inventors that one reason for the poor performance was that such open-cell pillar-array sensor structures allow multidirectional liquid wetting under increased pressure. To restrict the wetting of the liquid-solid-gas three-phase contact line unidirectionally, a closed-cell design is chosen in example embodiments of the present invention, A hexagon-array structure is chosen in a preferred example embodiment for optimal air accommodation within a given volume and minimal electrode exposure at the top. The results showed that the repeatability across cycles was greatly improved for the closed-cell sensors according to example embodiments. Note that other shapes of closed-cell columnar chambers, e.g. round, also help to improve the repeatability across cycles. Returning to FIG. 2C, surfaces I-IV with different advancing and receding contact angles by tuning the surface roughness and surface energies were investigated. The modifications and wetting properties were obtained using hexagon-array devices as shown in FIG. 2F without pillars for ease of fabrication and are summarized in Table 1. It is noted that the data presented in table 1 was obtained for hexagon-array devices without pillars for ease of fabrication, as shown in FIG. 2F.

TABLE 1

| | Modification | | | Contact | | |
|---|---|---|---|---|---|---|
| | on Hexagon- | Ad- | Forward | Angle | | Perfor- |
| | Array | vancing | Thres- | Hys- | Backward | mance |
| Sur- | Working | Angle | hold | teresis | Threshold | Hysteresis |
| face | Electrode | (°) | (kPa) | (°) | (kPa) | (%) |
| I | [symbol] | [symbol] | [symbol] | [symbol] | [symbol] | [symbol] |
| II | [symbol] | [symbol] | [symbol] | [symbol] | [symbol] | [symbol] |
| III | [symbol] | [symbol] | [symbol] | [symbol] | [symbol] | [symbol] |
| IV | [symbol] | [symbol] | [symbol] | [symbol] | [symbol] | [symbol] |

[symbol]

indicates data missing or illegible when filed

As shown in Table I and FIGS. 2G to J, example embodiments exhibit an advancing contact angle of the liquid under increasing pressure on the liquid that is greater than a static contact angle for water on a hydrophobic surface (which may be more than 65° or more than 90° according to various definitions) and less than 160°, less than 135°, less than 115° and less than 112°, respectively. Also, the contact angle hysteresis between advancing contact angle of the liquid under increasing pressure on the liquid and receding contact angle of the liquid under decreasing pressure on the liquid of less than 75°, less than 40°, less than 30° and less than 1°, respectively.

The differences in advancing angle and contact angle hysteresis on differently treated surfaces are due to the contact line being pinned by varying degrees of surface roughness. The high roughness of surfaces III and IV, induced by polyaniline (PAni) nanowires, significantly enhances contact line pinning, as shown in FIGS. 2G and H. Moreover, such rough surfaces incur irreversible Cassie-Baxter to Wenzel transitions under liquid pressure, further increasing advancing angles and contact angle hysteresis after applying pressure.

As the surface roughness reduces to the nanoscale (surface II), the advancing angle and contact angle hysteresis reduces accordingly, as shown in FIG. 2I. Upon silicone oil application facilitated by the porous PAni nanowires providing a larges surface area, surface I became molecularly smooth and ultra-slippery, as shown in FIG. 2J resembling slippery liquid infused porous surfaces (SLIPS) [11, 12]. This led to a significant reduction in contact line pinning, resulting in smaller advancing angle and negligible contact angle hysteresis, as shown in FIG. 2K. With increasing roughness, the four surfaces exhibited an increase in advancing angle and contact angle hysteresis.

Clear trends in interfacial wetting properties on sensing performance were observed: the larger the advancing angle, the higher the forward threshold, as shown in FIG. 2L; the larger the contact angle hysteresis, the higher the backward threshold and the hysteresis of devices, as shown in FIGS. 2M and N. FIG. 2O shows a graph illustrating the relationship between simulated contact area changes in hexagon-array devices (169 hexagonal chambers per device; side length, 40 μm) treated with surfaces I-IV, and measured capacitance changes of the corresponding devices, which are all linear (R²>0.99). Data are mean values±s.d. (five samples).

Advancing angle and contact angle hysteresis of different types of surfaces (I-IV) without applying liquid pressure and after applying liquid pressure (10 kPa) for 5 min were also investigated. For the surfaces with large surface roughness (Surface III and Surface IV), both the advancing angle and the contact angle hysteresis increased after applying pressure, but there was no such trend for smooth surfaces (Surface I and Surface II).

It is noted that the present invention is not limited to the use of the surfaces described above. Instead, in various embodiments other substantially friction-less surfaces for the 3D electrode structures, may be used, including solid and quasi-solid state material surfaces. In that regard, in various example embodiments, other surfaces that exhibit characteristics similar to those as shown in FIGS. 2G-O can be used based on different material(s) and/or different surface treatment(s), as will be appreciated by a person skilled in the art. For preferred embodiments, the contact angle hysteresis may be less than 10°, preferably less than 5°, and more preferably less than 3°.

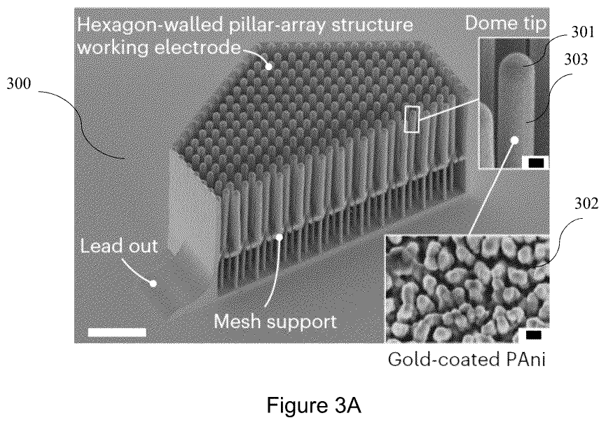

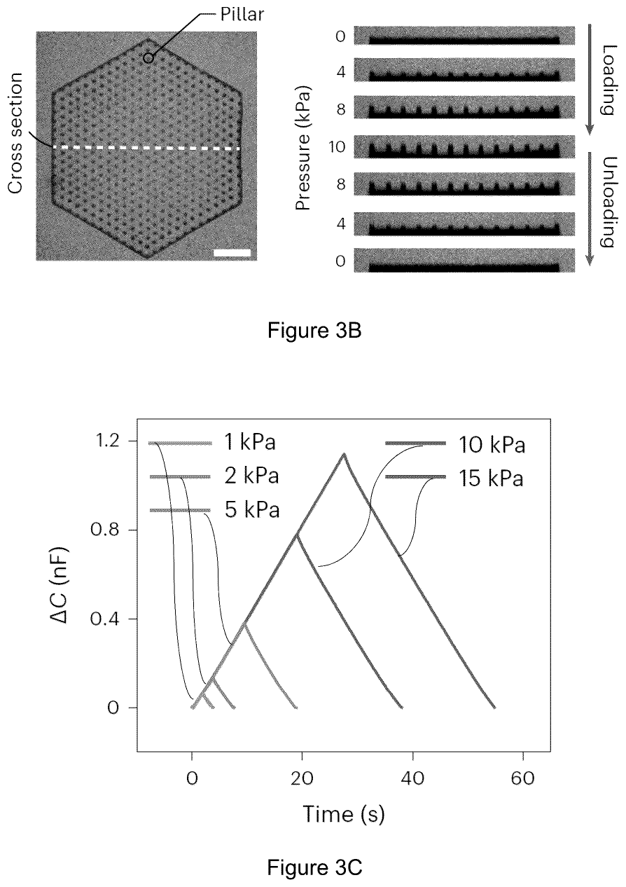

To further address the threshold effects and yet achieve high linearity, a hexagon-walled pillar-array structure was chosen in a preferred embodiment, as shown in FIG. 3A, which shows Scanning electron microscopy (SEM) image of the 3D-printed hexagon-walled pillar-array device 300 (in cross-section; scale bar, 200 μm), the pillar 303 with dome-shaped pillar tip 301 (scale bar, 10 μm) and the nanostructures 302 ((120 s PAni)/Cr/Au/ODT self-assembled monolayers (SAMs), 400 nm thick in total) on the pillar e.g. 303 surface (scale bar, 100 nm).

A domed tip pillar array was adopted in a preferred embodiment of hexagon-walled pillar-array device, to mitigate the surface tensional force when pillars penetrate the water surface under low loading pressure conditions. This mitigation is manifested by reducing the length of initial contact line and altering the direction of surface tensional forces.

The resistance to spreading of liquids at edges with smooth transitions (e.g. round edges of dome tips) is much smaller than at sharp edges. Once the devices are submerged in water, the contact line on the dome tip is more prone to move under low pressure. When the water surface comes into contact with the curved dome tip under external low pressure, the contact angle, which is ideally equivalent to 180 degrees, means that the contact line cannot be maintained in equilibrium. The contact line will move downward in search of a position where the contact angle is at least reduced to the advancing contact angle. Due to the specific shape of the curved dome, the reference plane for evaluating the contact angle is the tangent plane at the location of the contact point that moves as the contact line moves along the dome surface. In addition, since the advancing angle of Surface I is about 111 degrees, the contact line reaches a force balance only when it moves to a position close to the pillar cylindrical surface when no additional liquid pressure was applied. In actual wetting scenarios, water spreads from one side of the device to the other, and the liquid front exerts additional pressure on the pillars during the spreading process, causing the pillars to penetrate deeper. Note that the length of the contact line also increases as it moves downward, which is determined by the hemispherical shape of the dome. Thus, during the wetting process of the contact line on the dome, both the initial inclination angle of the reference plane and the length of the contact line are conducive to reducing the surface tension resistance when the dome tip “pierces” the water at small pressures.

In contrast, for flat tips, since the top flat surface shows 90-degree angle with the pillar cylindrical surface at the top edge, the initial contact angle, taken with the pillar cylindrical surface as a reference, is 90 degrees. The contact line length is initially the perimeter of the pillar, and the contact line is pinned at the top edge until the external pressure pushes the water to achieve an advancing angle, at which point the contact line starts to move. As an intuitive comparison, when the external pressure is gradually increased, the direction of the liquid surface tensional force at the contact line on the flat tip pillar has a larger angle with the horizontal plane when the contact line first reaches the advancing contact angle. This results in a greater vertical force component of the surface tensional force, thus leading to greater resistance to water wetting downward, leading to greater forward threshold.

Hence, the devices with dome tips do essentially not exhibit a forward threshold, while the devices with flat tips show a threshold of 1.32±0.37 kPa.

Implemented with the ultra-slippery surface I, the preferred embodiment with dome-shaped pillar tips can achieve a near zero-threshold, hysteresis-free and high-linearity pressure-sensing performance, as shown in FIG. 2B. Confocal microscopy images show the wetting process in such a device underwater at different pressures, as shown in FIG. 3B. The well-overlapping cyclic readings denote excellent performance consistency across different pressure ranges, see FIG. 3C. A pillar array in a hexagon-walled structure minimizes the forward threshold, according to the preferred embodiment. In contrast to use of an empty hexagon chamber, for which initial small pressures only cause the liquid-air interface to sag inward slightly, without increasing the liquid-electrode contact area, a pillar array in a hexagon-walled chamber according to the preferred embodiments advantageously enables an increase in the wetting contact area and corresponding capacitance from pillars under this small pressure-induced ‘saggy’ liquid-air interface.

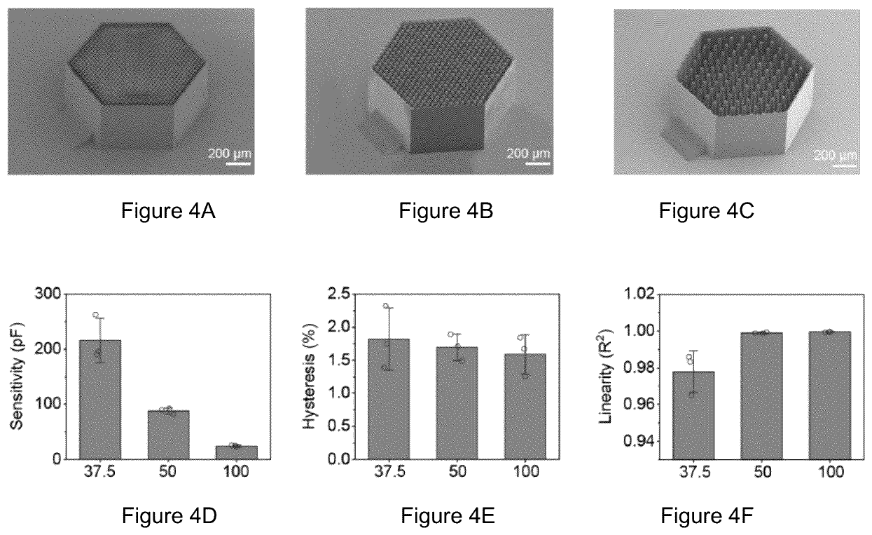

The pillars are designed with domed tips and small diameters (25 μm) in a preferred embodiment, to further minimize contact line movement resistance on pillars by reducing the initial contact line length and altering the surface tensional force direction, as described above. This advantageously allows these tips to ‘pierce’ into the liquid-air interface at small pressures. Comparing device performance across different pillar densities revealed an optimal pillar spacing of 50 μm, as shown in FIGS. 4A-C.

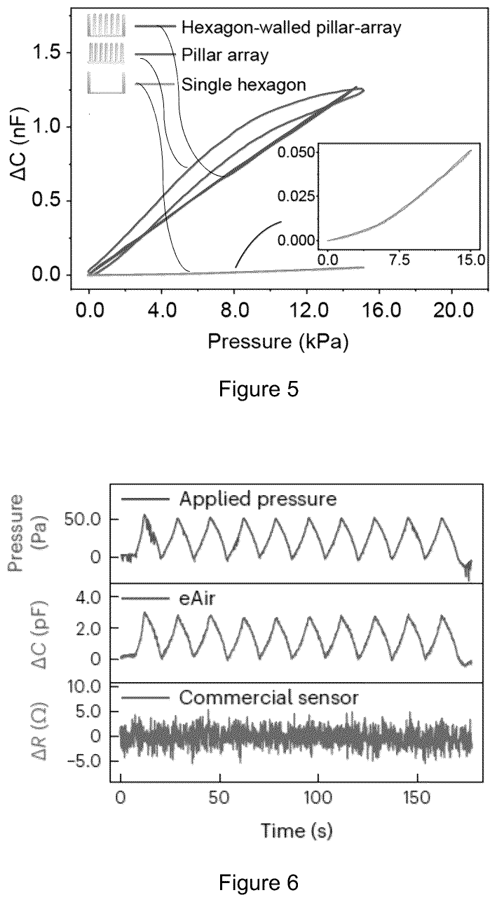

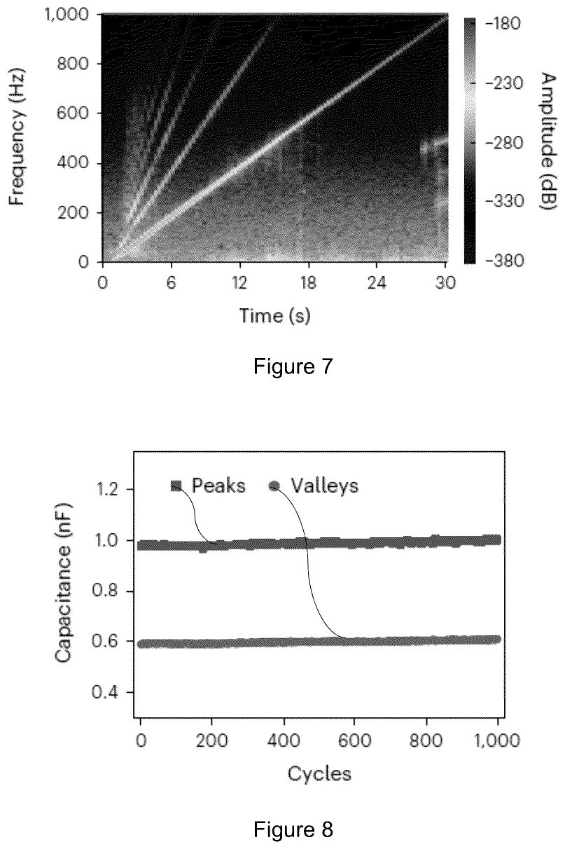

Accordingly, the iteration of structure optimization for a preferred embodiment involved three primary structures: open-cell pillar-array, closed-cell hexagon-array and their combination. FIG. 5 shows significant enhancement of the performance of example embodiments with the synergy of hexagon-wall and pillar-array structures. This structure design enabled to achieve a linear pressure-capacitance response with negligible forward threshold despite the absence of an advancing angle close to 90°. The eAir sensor according to a preferred embodiment can measure small pressures with very high signal-to-noise ratios (SNR) compared to a commercial in vivo pressure sensor, as shown in FIG. 6. It is noted that even after employing a Wheatstone bridge to mitigate noise, the commercial sensor's SNR remained lower. This threshold-free feature also enables the response to small dynamic pressure changes, such as underwater acoustic vibrations up to 1 kHz, as shown in FIG. 7.

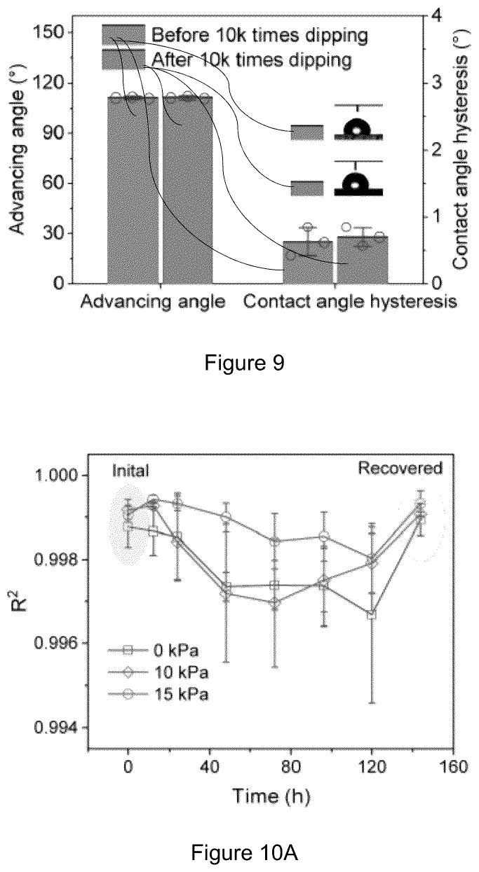

In terms of stability, eAir sensors according to a preferred embodiment demonstrated a minimal variation of 1.01% during 1,000 loading-unloading cycles of 0-5 kPa, as shown in FIG. 8. The ultra-slippery surface remained stable with negligible changes in advancing angle and contact angle hysteresis after 10,000 dipping cycles in a phosphate-buffered saline (PBS) solution, as shown in FIG. 9, highlighting the excellent lubricant locking ability of surface I nanostructures used in the preferred embodiment.

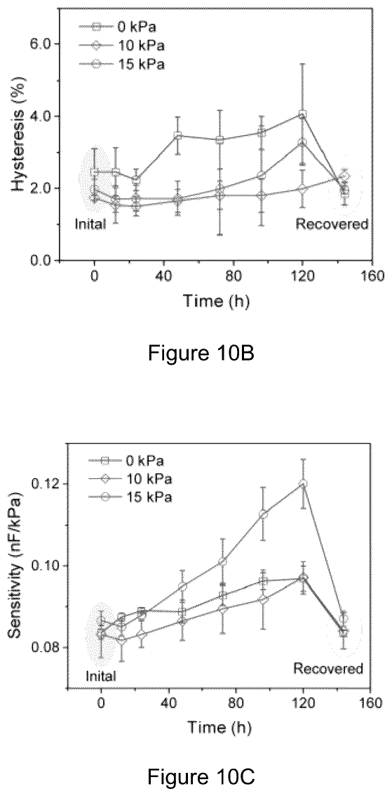

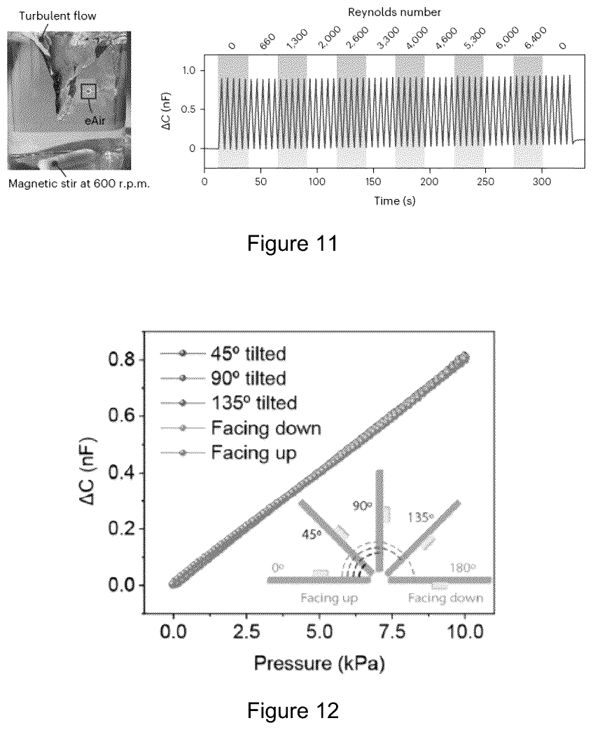

Moreover, eAir sensors according to a preferred embodiment sustained functionality after 5 days under constant pressures, as shown in FIG. 10A-C, with performance restorable to its initial state by reapplying the lubricant. The sensitivity was also consistent across several eAir sensors according to the preferred embodiment (79.1±4.3 pF kPa-1) despite initial capacitance variance due to differences in the exposed wiring area after electrode insulation. Notably, the eAir sensor according to a preferred embodiment functioned stably even in a high turbulent flow liquid environment with Reynolds number ~6,400, see FIG. 11. Device performance testing in the turbulent flow was done by using a magnetic stirrer (Wiggens digital hotplate/stirrer) to drive a stir bar (4 cm in length) to control the stirring speed in the range of 0-1,000 r.p.m., and simultaneously the pressure was cyclically changed between 0 and 10 kPa. To further test the stability, an additional setup was built where the sensor was placed under rotations (12 rpm) to face turbulent flow in various directions. The results obtained from this setup were consistent with the data presented FIG. 11 which reinforces the stability of the device's performance under the turbulent flow. In addition, the sensors were further tested inside a turbulent flow without cyclic pressure. At Reynolds number above 6,400, the high turbulent flow generates air pockets and voids which appear as bubbles in water due to the intense mixing of fluid. The bubble latched onto the sensor and caused a sudden capacitance drop and breakdown.

The capacitance varied with external hydrostatic pressure stably under turbulence induced by different stirring speeds. The eAir sensor according to a preferred embodiment was also unaffected by gravity as its internal spacings were much smaller than the capillary length (2.71 mm at room temperature), thus it maintained consistent performance across tests with different angled orientations, as shown in FIG. 12, noting that due to the overlap between the measurements, the different curves are indistinguishable in FIG. 12.

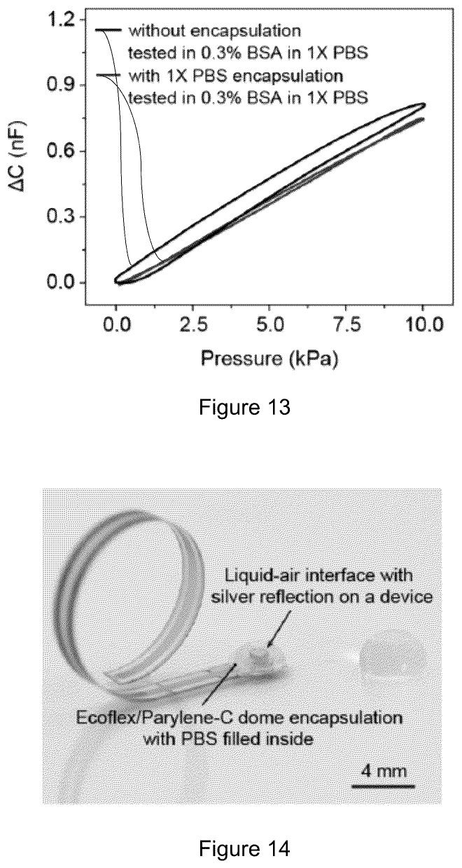

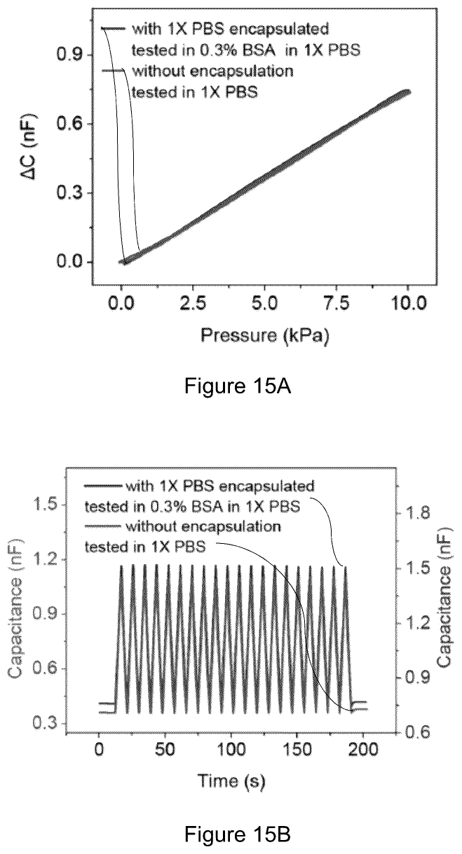

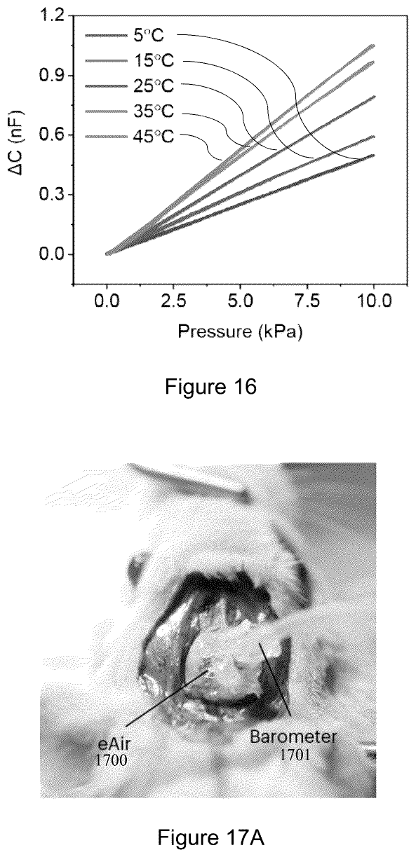

It was found that complex liquid environments (for example, bioliquids) can affect sensing behaviour, as shown in FIG. 13. To expand usage scenarios of the eAir sensor according to example embodiments, the sensor device was encapsulated in a thin Ecoflex/parylene dome film filled with 1×PBS solution to isolate it from external environments, as shown in FIG. 14. The encapsulated device showed unchanged performance except for an increase in the initial capacitance as shown in FIGS. 15 A, B. Despite increasing sensitivity with temperature, the eAir sensor according to a preferred example embodiment preserved high linearity and low hysteresis across different ambient temperatures, as shown in FIG. 16.

Animal test of intracranial pressure (ICP) sensing using sensors according to example embodiments.

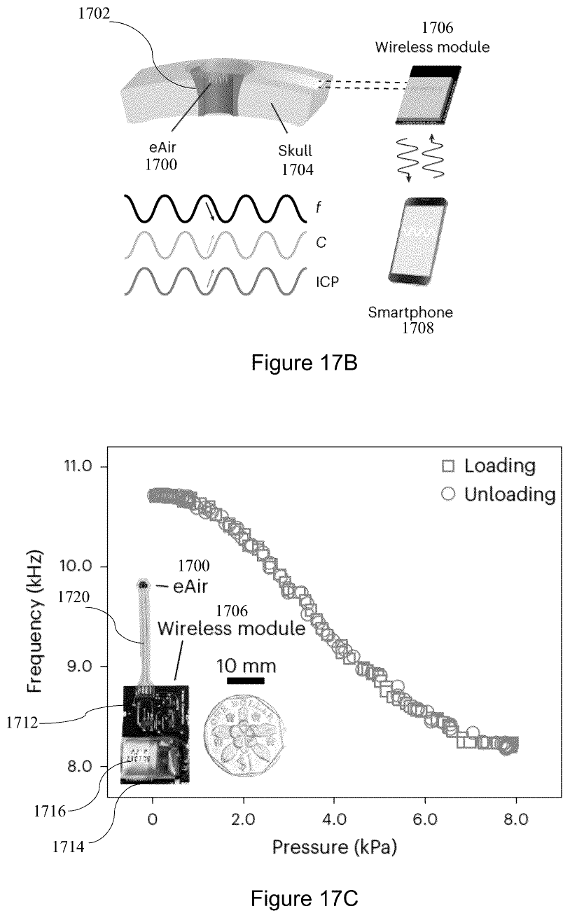

Male Sprague-Dawley rats (5-6 months old, 350-500 g, InVivos) were used to demonstrate in vivo ICP monitoring using sensor according to example embodiments. Rats were anaesthetized with isoflurane (1-3%) inhalation and intraperitoneal injection of ketamine/xylazine (75/10 mg per kg (body weight)) and carprofen (5 mg per kg (body weight)). Fur was sheared from the top of the animal's head, and the head was placed in a stereotactic apparatus (David Kopf Instruments) attached to an oxygen and isoflurane flow. Lubricant eye ointment (Alcon) was applied. After disinfection with iodine and isopropyl alcohol, a midline scalp incision was made, and two burr holes (~2.5 mm in diameter) were created at bilateral parietal bones by craniotomy using a high-speed drill (RWD Life Science). An encapsulated eAir sensor 1700 on flexible polyimide substrate with a wireless module according to an example embodiment and a sterilized silicone tube 1701 with a commercial barometer (DLHR-L60D, Amphenol All Sensors) were attached to the left and right burr holes and sealed with dental cement (Parkell), respectively, as shown in FIG. 17A. All experiments conformed to the Guide for the Care and Use of Laboratory Animals published by the National Institutes of Health, USA, and the protocol was approved by the Institutional Animal Care and Use Committee, National University of Singapore.

ICP is the pressure created by fluids such as cerebrospinal fluid inside the skull and on the brain tissue. Normal ICP ranges between 5 and 15 mmHg (660-2,000 Pa), a sustained pressure above 20 mmHg (2,660 Pa) is considered pathological, and severe ICP of >40 mmHg (>5,330 Pa) indicates life-threatening intracranial hypertension37. The small size and biocompatibility of the eAir according to example embodiments advantageously enable continuous ICP monitoring. Wireless monitoring of ICP in a rat model with an encapsulated eAir sensor attached to a patterned flexible polyimide substrate was demonstrated. With reference to FIG. 17B, the eAir sensor 1700 and the barometer (not shown) were mounted into respective burr holes e.g. 1702 on a rat skull 1704. To enable wireless readout of the ICP, the eAir sensor 1700 was connected to a wireless module 1706 to continuously broadcast ICP signals to a nearby mobile device 1708. The eAir sensor 1700 was encapsulated by an Ecoflex dome with 1×PBS filled inside the dome.

An oscillation circuit converted the pressure fluctuations to a digitizable frequency signal. As shown in FIG. 17C, the oscillation circuit was assembled on a rigid printed-circuit board 1712 (PCB, 18×25 mm) by microsoldering (NAE-2A, JBC) three surface-mount thin film resistors (CR0201-FW-1001GLF, CR0201-FW-2001GLF, and CR0201-FW-49R9GLF; Bourns) and a precision amplifier (AD8538WAUJZ-R7, Analog Devices). The oscillation circuit was further connected to a Bluetooth microcontroller unit 1714 (ESP32-WROOM-32D, Espressif Systems) to form the wireless module 1706, noting that in FIG. 17C the Bluetooth microcontroller unit 1714 is hidden underneath a lithium polymer battery 1716 (100 mAh, 3.7 V) with a voltage regulator (NCP167AMX330TBG, Onsemi) used to power the whole wireless module 1706. An anisotropic conductive tape (7303, 3M) was used to connect the polyimide substrate 1720 with eAir sensor 1700 to the PCB 1712. The electronics was encapsulated by surgical silicone adhesive (Kwik-Sil Adhesive) for protection. The frequency response was read by the internal pulse counter of ESP32 and sent to an Android smartphone as the mobile device 1708 (see FIG. 17B) through Bluetooth. The oscillation frequency was also characterized by a mixed domain oscilloscope (MDO3012, Tektronix).

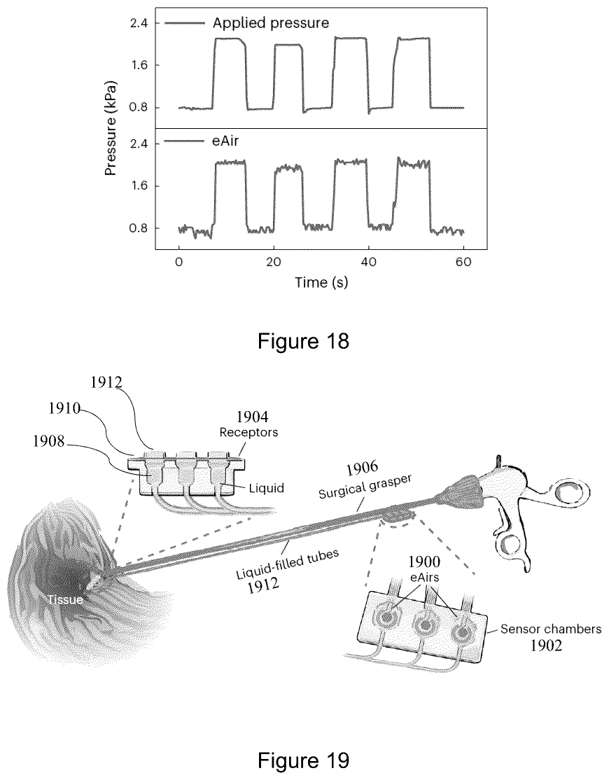

The frequency response of the oscillation circuit was calibrated to determine the relationship between frequency and pressure, as shown in the graph in FIG. 17C. The wireless ICP sensor output agreed well with the barometer, as shown in FIG. 18, verifying the feasibility and effectiveness of an implanted ICP sensor according to an example embodiment under complex in vivo conditions. The eAir sensor's minimally invasive nature and precise dynamic response make it a promising candidate for future ICP and other biofluid pressure monitoring.

Integration of eAir Sensors with Laparoscopic Surgical Grasper According to Example Embodiments

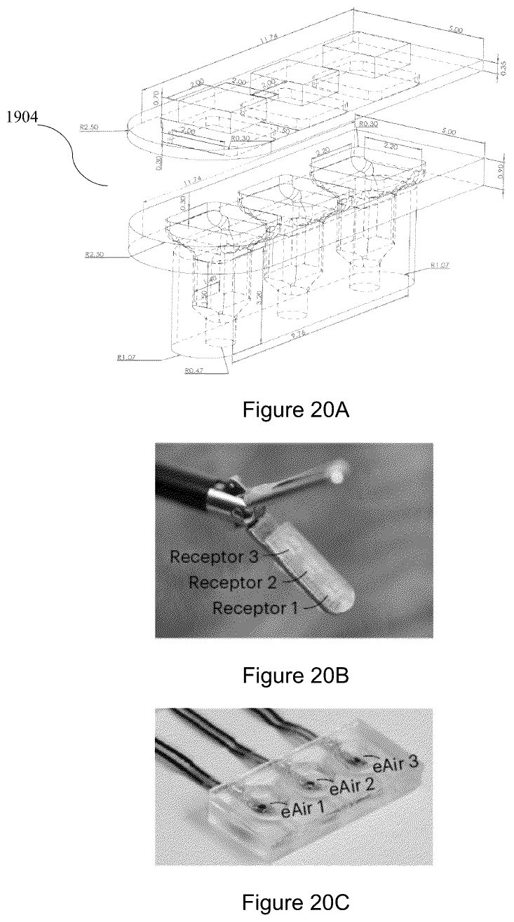

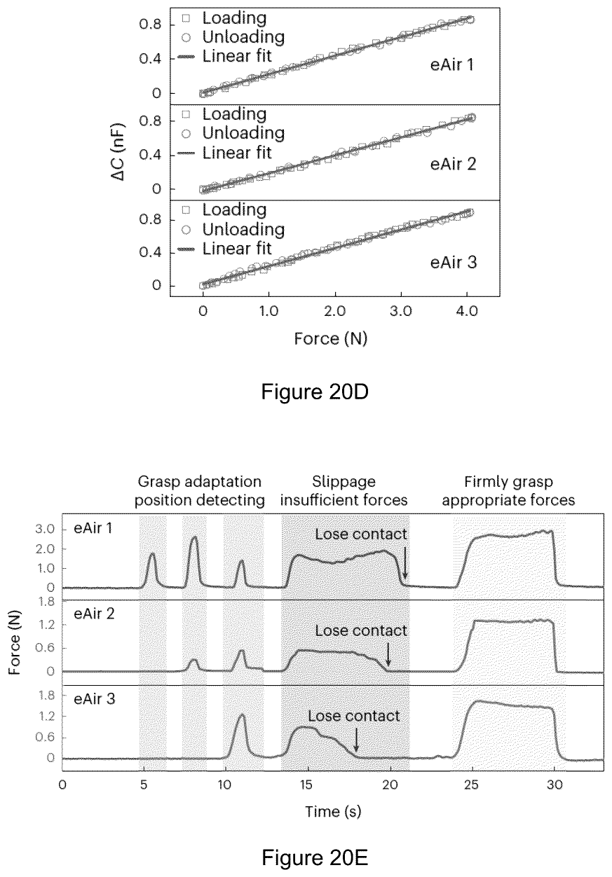

With reference to FIG. 19, three eAir sensors 1900 were encapsulated in three identical closed chambers 1902, and three corresponding receptors 1904 forming three tactile pixels were configured and mounted into one jaw of a fenestrated grasper 1906 (5 mm in diameter) to assess tactile perception. The eAir sensors 1900 were immobilized in the encapsulation chambers 1902 with Ecoflex. The receptors 1904 were designed with rigid chambers 1908 covered by elastic film 1910 with protrusions 1912 for sufficient deformation under external force. The eAir sensor encapsulation chambers 1902 and receptors 1904 were designed with SolidWorks and printed with a stereolithography-based 3D printer (Formlabs 3B+, Formlabs). The elastic top film 1910 and the rigid chambers 1908 of the compressible receptors 1904 were printed with commercially available flexible 80 A and BioMed Clear (Formlabs) resins, respectively. The dimensions of the receptors 1904 are shown in FIG. 20A. Photographs of the receptors on the jaw and of the eAir sensors are shown in FIGS. 20B and C, respectively. Returning to FIG. 19, the chambers 1902, 1908 were connected with PTFE tubes 1912 (inner diameter, 0.3 mm; outer diameter, 0.6 mm, length, 25 cm) to conduct the liquid pressure between the receptors 1904 and corresponding eAir encapsulation chambers 1902. The tactile feedback system was filled with 1×PBS solution, except inside of the pillar array of the eAir sensors 1900 due to the hydrophobic nature of the device structure, as described in detail above, and encapsulated under liquid to avoid undesirable air bubble generation. It is noted that no Ecoflex dome is used around the eAir sensors 1900 in this example embodiment. The various interconnects were sealed with BioMed Clear Resin and further cured to prevent liquid leakage under increasing pressure.

To create a calibration matrix, a linear stage-driven digital force gauge (Mark-10 MR03-5) with a probe (2 mm×2.5 mm) was used to apply a normal force (0-4 N) to the top of the protrusions 1912 of the compressible receptors 1904. To check the tendency of the capacitance change under the influence of an external force, the eAir sensors 1900 were characterized by using three portable impedance analysers (Digilent Analog Discovery 2). The three eAir sensors 1900 showed a linear response to the external force. The eAir sensors 1900 were easily calibrated using the calibration matrix due to their linear characteristics. To verify the performance, the grasping forces of the sensorized laparoscopic surgical grasper 1906 were measured. Data from the three eAir sensors 1900 were recorded simultaneously while grasping the reference ‘tissue’ part, which was made from dyed Ecoflex 00-50 (Smooth-On). The data were converted into force using the calibration method mentioned above.

Accordingly, the eAir sensors 1900 can readily furnish laparoscopic tools with tactile sensitivity. Over 13 million laparoscopic surgeries are performed annually worldwide. A high rate of complications is caused by improper grasping forces when using laparoscopic graspers. Excessive force, for example, was found to be the cause of 11.3% of consequential and 19.0% of inconsequential grasper-related errors in a study of laparoscopic cholecystectomies. However, it has been challenging to incorporate tactile sensing due to large sizes, integration complexity and performance limitations. The working principle of the eAir sensors 1900 according to example embodiments allows for remote force sensing because it relies on fluids. In an example embodiment of the sensorized laparoscopic surgical grasper 1906, the eAir sensors 1900 can still remarkably deliver forces with high linearity (R²=0.99646±0.00112), low hysteresis (5.66±1.38%) and high sensitivity (215.2±5.6 pF N⁻¹), as shown in FIG. 20D.

Returning again to FIG. 19, the application of real-time grasping force monitoring during laparoscopic surgery was simulated using the sensorized laparoscopic surgical grasper 1906. The tissue was tentatively grasped by advancing the grasper 1906 gradually to achieve proper contact. The precise contact positions were revealed by the eAir sensors 1900 readings, allowing to adjust the grasping position accordingly. Second, the tissue was gently grasped and pulled. The readings displayed a successive loss of contact pressure, indicating tissue slippage due to insufficient grasping force. Such slippages could result in patient trauma during actual laparoscopic procedures. Finally, the tissue was grasped with a greater force and the grasper was shaken. Minor variations in the eAir sensors 1900 readings were observed without any contact loss, indicating firm grasping of tissue by the jaws and tissue slippage was avoided, as shown in FIG. 20E. This test demonstrates the potential of the eAir sensors according to example embodiments to provide tactile feedback in laparoscopic procedures.

### Testing of Sensors According to Example Embodiments

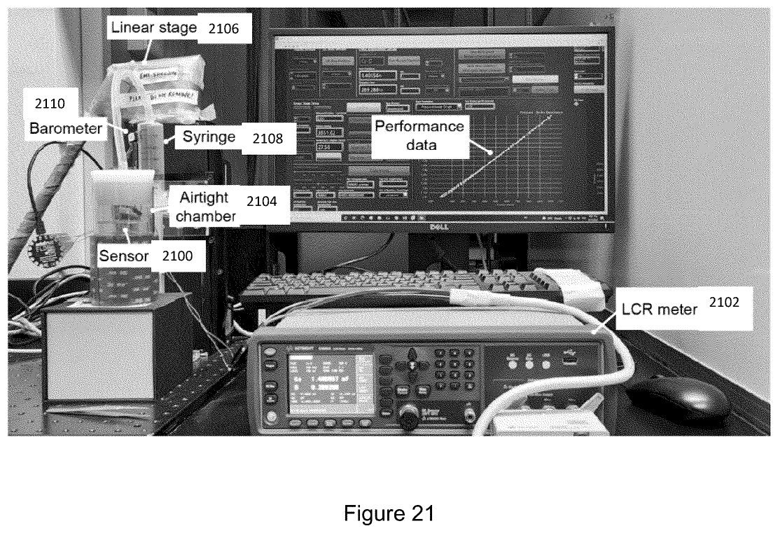

FIG. 21 shows the test setup for the pressure sensors according to example embodiments.

The sensors 2100 were characterized using an LCR meter 2102 (Keysight E4980A) in varying pressure. The LCR meter 2102 was set to work at the Cs-D (Series Capacitance-Dissipation factor) function with a test signal level of 100 mVrms (100 kHz). The applied voltage was too small to induce electrowetting, which was confirmed by observing the device under a confocal microscope (Olympus FV3000) with an applied AC voltage of 100 mVrms. The sensors 2100 were encased in 1×phosphate buffered saline (PBS, BioWhittaker) in a custom airtight chamber 2104. It is noted that in order to minimize the pressure difference between the air pressure in the airtight chamber and the pressure applied to the sensors, i.e. the pressure caused by the liquid itself (depth), one a small drop of 1×PBS was used to encase the 3D structure, thus the air pressure in the airtight chamber was consistent with the pressure applied to the sensors.

The periodic loading and unloading pressure were achieved by pumping air into and out of the chamber 2104, i.e. in the space above the 1× phosphate buffered saline, using a linear stage 2106—driven syringe 2108 with a high-precision commercial barometer 2110 (DLHR-L60D, Amphenol All Sensors Corporation) as feedback. The pressure changing rate was control by the speed of the linear stage 2106. For the sound frequency response test, a loudspeaker (not shown) was used to generate vibrations in the frequency range of 0-1 kHz.

For in vivo sensor characterization, the LCR meter 2102 was used to record the ground-truth resistance changes, and the sensor was also tested with its official evaluation module (with a Wheatstone bridge and signal conditioning circuitry to output calibrated pressure readings).

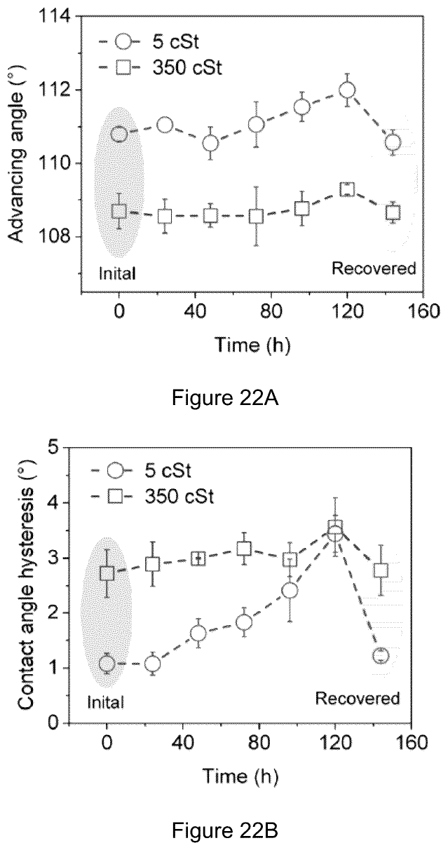

To assess the stability of the ultra-slippery surface (Surface I), tests of its stability were conducted under cyclic liquid shearing and long-term immersion in 1×PBS solution. The advancing angle and contact angle hysteresis showed negligible changes after 10,000 dipping cycles in a phosphate-buffered saline (1×PBS) solution, indicating its good stability (see FIG. 9. Further, glass slides coated with Surface I (coated with 5 cSt silicone oil) were fully immersed in a 1×PBS solution for 5 days to monitor the changes in surface wetting properties. The results show that the advancing angle and contact angle hysteresis both increase with the immersing time. However, it is important to note that the increase was not excessive but within a reasonable range as shown in FIGS. 22A and B. The advancing angle increased by only 1.20±0.11°, and the contact angle hysteresis remains within 4°. This slight degradation in the slippery performance could be attributed to the loss of oil, which occurs due to the slow diffusion of oil into the PBS solution during prolonged immersion (for 5 days).

The variations in contact angles in a prolonged period might contribute to the performance drift of eAir according to example embodiments. To evaluate the stability of the eAir sensors over time when immersed underwater, tests of devices immersed in 1×PBS solution with constant pressures of 0 kPa, 10 kPa, and 15 kPa, separately, were conducted. The devices were monitored for a period of 5 days to observe any changes in performance, especially in terms of linearity, hysteresis, and sensitivity. The device was still functioning properly on the 5th day, but the device performance degraded over time. Specifically, a decrease in linearity and an increase in hysteresis and sensitivity were observed. The degradation in device performance observed during the stability is within expectations, because the properties of the super-slippery surface can deteriorate over time, and the air inside the device also slowly dissolves into water over time. As previously mentioned, the loss of oil from the ultra-slippery surface degrades its frictionless properties, causing a delay in the movement of the multiphasic contact line. In addition, the loss of oil also reduces the thickness of the dielectric layer, leading to an increase in capacitance.

Despite the observed degradation, the performance variations were small and acceptable until the 48-hour mark, with a sensitivity change of less than 10%, negligible linearity change (with R2 changing within 0.1%), and hysteresis remaining within 2.5%. Beyond 48 hours, the performance variations continued to increase, but the device still functioned properly without any performance breakdown. This indicates that there was still sufficient air within the device that was not dissolved completely. Theoretically, if there is a high volume ratio between water and air, the water-air interface may easily break down, resulting in a transition from Cassie state to Wenzel state. In the testing of example embodiments described herein, there are two scenarios:
1. Performance testing in the airtight chamber. First of all, the PBS solution was stored in an atmospheric environment (ample amount of air), was already air-saturated at atmospheric pressure. During device testing, the device is placed inside a large airtight chamber and a small drop of air-saturated PBS solution is applied to cover/encase the device. The air inside the airtight chamber is ample compared to the air trapped within the device. Since the PBS is already saturated with air, no more air dissolves into the PBS solution without increasing pressure. When pressure increases, the larger contact area between the applied droplet and the large volume of air in the airtight chamber, compared to the smaller contact area between the air inside the device and the droplet, leads to a faster saturation of the droplet with the airtight chamber's air. The rapid saturation hinders any significant air dissolution from within the device.
2. Device with Ecoflex dome encapsulation. In this case, since the PBS solution used is already saturated with air, the air trapped inside the device does not continue to dissolve into the PBS solution under atmospheric pressure. When the pressure increases, the small volume of PBS solution encased in the dome is insufficient to cause a significant dissolution of air from the trapped air within the device.

Moreover, reapplying oil to the structured surface in the device can allow the sensing performance to be restored to the initial state, with a sensitivity change of less than 1%, negligible linearity change (with R² changing within 0.1%), and hysteresis remaining within 2%. Therefore, the degraded performance of the eAir sensors according to example embodiments can be fully recovered. Even with encapsulated devices, reoiling is easy because the encapsulation film can be removed easily. Also, this process is simple and does not require any specialized equipment, making it feasible for users to perform on their own. At low pressure testing range, the sensor's stability according to example embodiments can sustain continuous measurement up to 5 days. By reoiling the device, a longer service life can be achieved. For the example application in grasper usage conditions and intracranial measurements, the current service life meets the necessary requirements, making the sensor according to example embodiments a viable and suitable option.

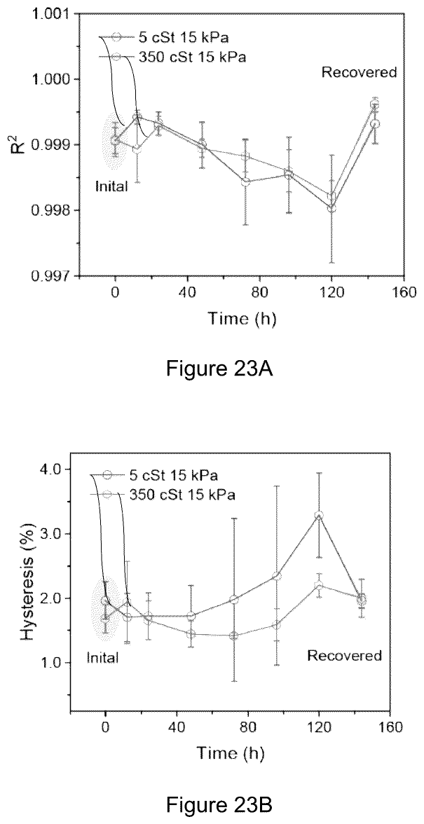

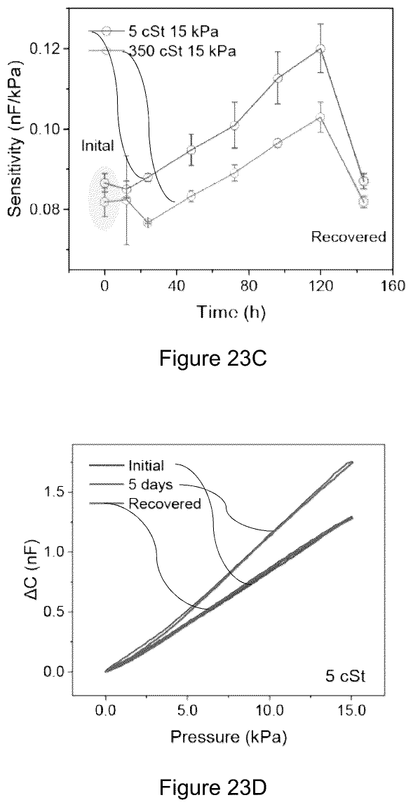

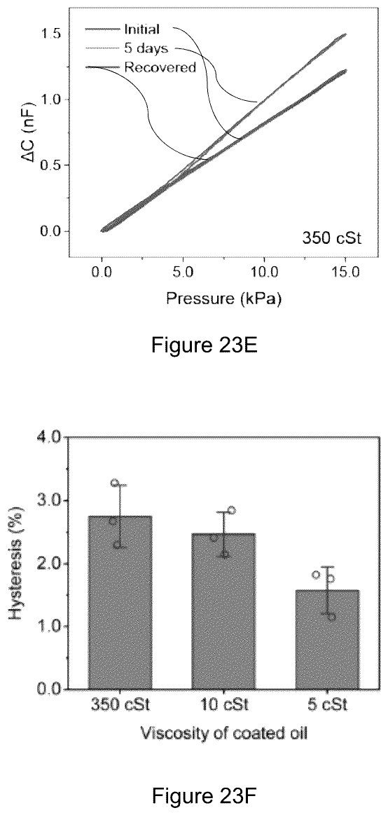

To further enhance the stability of the device in example embodiments, the viscosity of the silicone oil can be increased. While lower viscosity oil is desirable for achieving lower friction and higher contact line mobility, higher viscosity results in a lower oil loss rate and higher stability, making it suitable for creating a more stable ultra-slippery surface on the device. To evaluate the feasibility of the strategy, tests of devices coated with different viscosities of silicone oil under a maximum pressure of 15 kPa were conducted. Specifically, the sensing performance of devices coated with 350 cSt and 5 cSt silicone oil we monitored over time. The devices were immersed in 1×PBS solution and subjected to constant pressures of 15 kPa. As shown in FIGS. 22A and B, the 350 cSt silicone oil resulted in smaller surface property variation, especially for contact angle hysteresis. Additionally, as shown in FIGS. 23A-C, the improved surface stability led to smaller variations in linearity, hysteresis, and sensitivity when compared to the 5 cSt silicone oil, and hence in a more stable performance due to the lower oil loss rate of higher viscosity silicone oil, compare FIGS. 23D and E. Therefore, increasing the oil viscosity is an effective way to enhance the device stability according to example embodiments. The effect of viscosity of coated oil on hysteresis performance of the eAir is shown in FIG. 23F. The eAir sensors were coated with the same volume of oils (5 μL, 0.1% v/v silicone oil diluted in n-heptane) and tested at the same pressure. Data are represented as mean values±s.d. (3 samples) with corresponding data points overlapped.

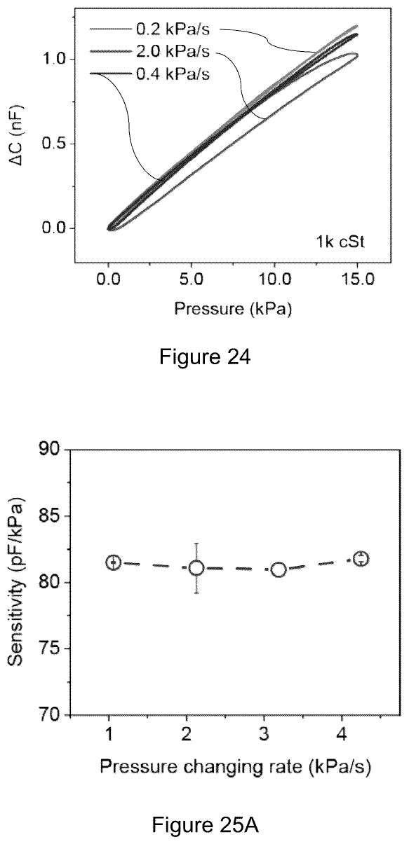

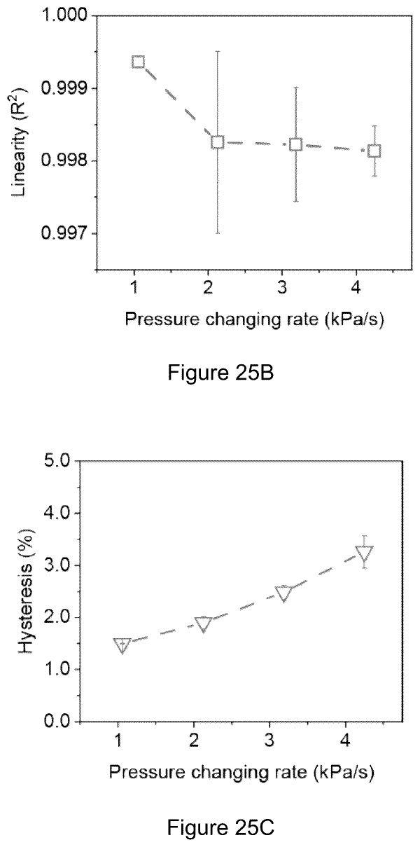

An eAir sensor according to an example embodiment with silicone oil of 1k cSt was also tested and it was found that the device can exhibit linear and low hysteresis performance at low pressure changing rates of 0.2 kPa/s, as shown in FIG. 24. However, the sensing performance was found to be greatly affected by different pressure changing rates. Differently, for devices coated with low viscosity silicone oil (5 cSt), as shown in FIGS. 25A-C, the sensitivity remained unaffected by varying rates, and the linearity and hysteresis showed a small deterioration with increasing pressure changing rates. Overall, the sensors according to embodiments with surface I showed good performance consistency under different pressure changing rates.

For the sensor performance recovery test, n-heptane was used to wash off the silicone oil from the device, followed by reapplying the oil and testing the device's performance. The effect of temperature on device performance was tested using a circulating water bath (CORIO refrigerated/heating circulator CD-200F) to control the temperature of the liquid environment.

The capacitance response of eAir under different temperatures is shown in FIG. 16 for the eAir with surface I, according to an example embodiment, and maintained linearity and low hysteresis performance over the 5° C. to 45° C. range. The eAir sensors were tested in 1×PBS with cyclic pressure applied. For the influence of temperature, the eAir sensors showed an increasing sensitivity with temperature, but still preserved high linearity and low hysteresis at different ambient temperature.

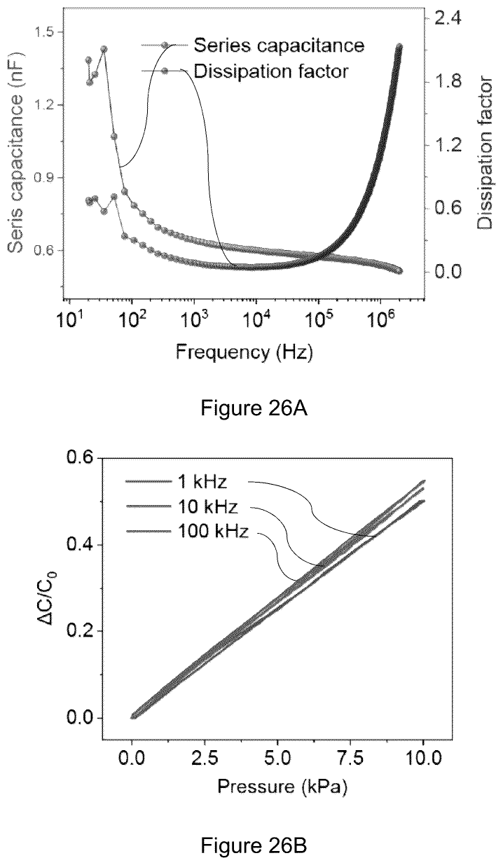

The effect of frequency on the eAir sensor with surface I performance was investigated with a test signal frequency sweep from 20 Hz to 2 MHz without external pressure applied, as shown In FIG. 26A. FIG. 26B shows the capacitance response of the eAir with surface I tested at different frequencies with cyclic pressure applied.

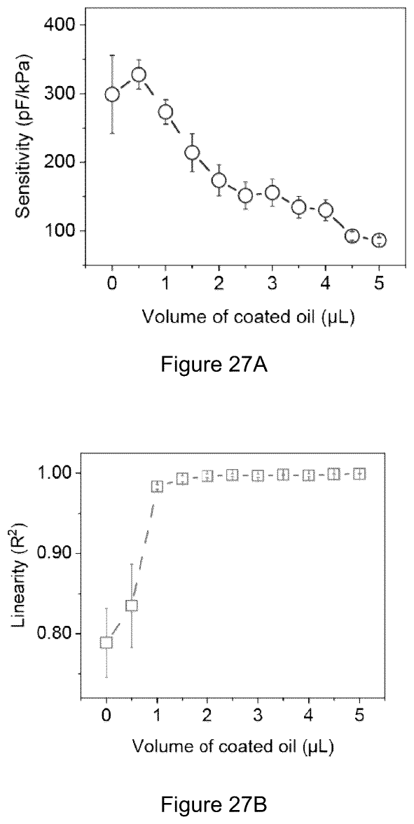

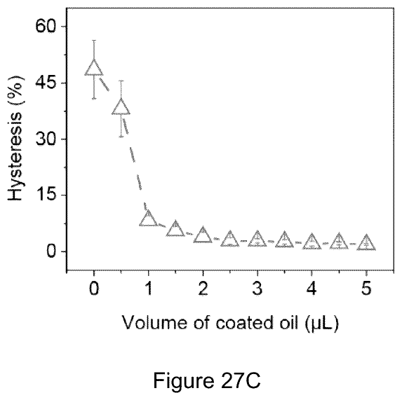

The effect of volume of coated diluted oil on sensing performance of the eAir sensors is shown in FIGS. 27A-C. Sensitivity, linearity, and hysteresis vary with the volume of coated diluted oil (0.1% v/v of 5 cSt silicone oil diluted in n-heptane). All three performance parameters tend to be optimal after 5 μL of the diluted oil was applied. Data are represented as mean values s.d. (5 samples).

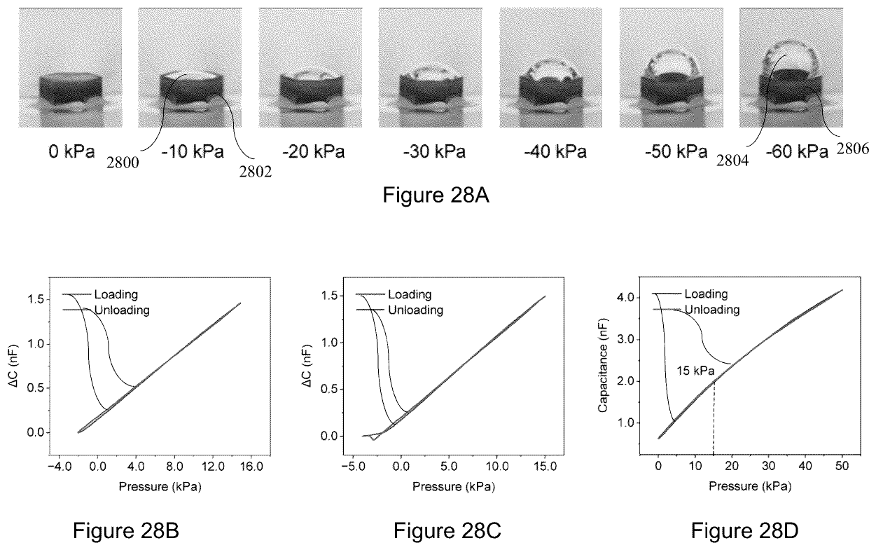

Sensors according to example embodiments under negative pressure and high pressure The air entrapped in the eAir sensors remains on the device under negative pressure as low as −60 kPa and does not detach. As shown in FIG. 28A, when subjecting the immersed eAir device to various negative pressures a noticeable bubble 2800 protrusion at the top of the eAir structure 2802 was observed when the pressure was reduced to −10 kPa. As the pressure decreased further, the size of the bubbles increased; however, the bubble's 2804 edge remained pinned to the top edge of the structure's 2806 wall even when the pressure reached −60 kPa, the lowest pressure the testing setup can achieve. This negative pressure is significantly lower than the operating range of 0-15 kPa for the eAir devices stated above.

Moreover, it was found that the eAir sensors maintain substantially the same sensitivity and linear response as in the ranges of 0-15 kPa even when pressure was as low as −2 kPa as shown in FIG. 28B. This is because, at zero pressure, the dome tip pillars were already partially penetrated in water. Under slight negative pressure, the water-air interface moves outward, but it does not detach from the pillars until the pressure falls below −2 kPa. However, when the pressure reached to −4 kPa, the water located at the central position of the structure started to detach from the pillars, generating an air bubble that is “out” of the inner structure and causing the device to cease exhibiting a linear response to pressure, as shown in FIG. 28C.

The eAir sensors according to example embodiments also respond to pressures exceeding the stated 15 kPa. As shown in FIG. 28D, the eAir sensors can respond to pressures as high as 50 kPa. It could potentially respond to even higher pressures, but due to the limitations of the testing setup's pressurizing capability, only up to a maximum pressure of 50 kPa was measured. The eAir sensors exhibited low hysteresis within 0-50 kPa and maintained a linear relationship between capacitance and pressure within the 0-15 kPa range, but as the pressure continues to increase, the sensitivity gradually decreases, exhibiting non-linearity. This is because as the pressure increases, the air becomes increasingly difficult to compress. The same change in pressure results in a smaller change in volume of air and a reduced change in the water-electrode contact area with increasing pressure.

### Definition of Hysteresis, Linearity, and Sensitivity

Hysteresis (H) is defined herein as the maximum absolute difference between the loading curve and the unloading curve at the same pressure as a percentage of the full range span (YFS). Linearity is quantified herein in two ways. One adopts the R-square (coefficient of determination) value of the linear fitting result using the Simple Linear Regression Model (computed by OriginPro software) with consideration of both loading and unloading. The other is to calculate the nonlinearity of the device performance curve. Here, the nonlinearity (L_{non.}) is expressed as a percentage of the full range span (YFS) occupied by the maximum value of the absolute value of the subtraction of the loading and unloading curves at the same pressure from the linear fit line.

Sensitivity is defined herein as the change in output value caused by a change in unit pressure. An ideal pressure sensor with high accuracy should have a linear, zero-threshold, hysteresis free, and sensitive response. Specifically, poor hysteresis and linearity can cause sensing errors and signal distortion, and such sensors operate with limited consistency in sensitivity over their working ranges, thereby needing additional pressure-output calibration.

### Capacitance Analysis of Sensors According to Example Embodiments

The capacitance (C) composition of the sensor according to an example embodiment and its equivalent circuit were illustrated in FIG. 2C, also showing the surface coating details between structural working electrode and liquid.

The device capacitance can be expressed as

$$C = \frac{1}{\frac{1}{{C}_{0} + \frac{{C}_{d . ct} {C}_{EDL . ct}}{{C}_{d . ct} + {C}_{EDL . ct}} + {C}_{d + air}} + \frac{1}{{C}_{counter}}} , \qquad ( Eq . 1 )$$

where C₀ is the interfacial capacitance of the non-active area, which is the area outside the chambers, i.e., the top area of the structural working electrode in contact with the liquid and the wires encapsulated with insulations (SU-8 and polyimide). In contrast, the active area is defined as the inner surface area of the chambers, where the dynamic wetting happens. Since the capacitance of each component follows the physical principle of the parallel plate capacitor, their values can be calculated using εA/d, where ε is the permittivity of the dielectric material, A is the area of the plates, and d is the equivalent distance between the two plates. The capacitance between the noncontacted area of the structural electrode and the liquid-air interface through the surface coating layer and air, C_{d+air}, can be ignored as its equivalent d is much larger than the other capacitors. C_{d,ct} and C_{EDL,ct} can be considered as an interfacial capacitance C_{ct}, i.e., the interfacial capacitance between the structural electrode and the liquid within the liquid-electrode contact area.

Eq. 1 can thus be simplified to,

$$C \approx \frac{{C}_{ct} + {C}_{0}}{1 + ( {C}_{ct} + {C}_{0} ) / {C}_{counter}} . \qquad ( Eq . 2 )$$

For Surface I, the dielectric layer is a composition of ODT SAMs and silicone oil, as PAni nanowires and the gold coated on the nanowires are conductive. The thickness of ODT SAMs is in few nanometers (thickness ≈2 nm, dielectric constant: ~2.1), and the silicone oil is infused between the PAni nanowire induced nanostructures. Therefore, the dielectric layer is ultrathin. The unit capacitance of a Surface I treated electrode was measured as 1.87±0.30 nF/mm².

For Surface II, the dielectric layer is the ODT SAMs. The unit capacitance of a Surface II treated surface was measured as 6.79±0.64 nF/mm².

For Surface III and IV, as PAni nanowires are conductive, the dielectric layers are the ODT SAMs and the air trapped between the surface nanostructures induced by PAni nanowires (gold coated). The unit capacitance of Surface III and Surface IV treated electrodes were measured as 7.05±0.16 nF/mm² and 7.98±0.54 nF/mm², respectively.

The unit capacitance of bare gold electrode was measured as 97.3±10.1 nF/mm2. The unit capacitance was measured using two identically treated electrodes with the same area (1 cm²) immersed in 1×PBS solution, 1 cm apart, with 100 mVrms 100 kHz voltage applied. It is noted that the measured capacitance decreases with the distance between the electrodes in the solution, while the distance between the structural electrode and the counter electrode of the actual device is much smaller than 1 cm, so the unit capacitance obtained here is smaller than the unit capacitance inside the eAir device.

Moreover, in all devices with different surfaces, the liquid-electrode contact areas on the structural working electrode are much smaller than the area of the counter electrode (3.2 mm²), which was confirmed by simulation results. Also, the capacitance of the non-active area can be divided into two parts: i) the exposed area of the top of the structural working electrode and part of the electrode area on the wall for wire leading out without encapsulation. This part is less than 0.6 mm², and it varies with the manual encapsulation of SU-8. The area has the same surface treatment as the device, thus the capacitance, C₀ is at the same order of magnitude of C_{ct} with consideration of both areas; ii) the area of the wires encapsulated with insulations (SU-8 and polyimide). As the insulations are very thick compared to the surface treatments, this part of capacitance can be ignored. Therefore, all devices with different surfaces follow the same relationship between capacitances: C_{counter}>>C_{ct}+C₀.

Therefore, C_{counter}>>(C_{ct}+C₀)/C_{counter} is much smaller than 1, and Eq. 2 can be further considered as,

$$C \approx {C}_{ct} + {C}_{0} . \qquad ( Eq . 3 )$$

C_{ct} can be calculated as

$${C}_{ct} = {ε}_{ct} {A}_{ct} / {d}_{ct} , {ε}_{ct} , \qquad ( Eq . 4 )$$

where ε_{ct} and d_{ct} are the equivalent permittivity and electrode distance between the structural electrode and the liquid-coating interface, Act is the contact area change induced by the liquid pressure between the liquid and the treated structural working electrode. Since for fixed surface treatment, the permittivity and distance are constants, one can define a constant k=εcₜ/d_{ct}. Therefore, one has the following equation for all devices with different surfaces,

$$C \approx {C}_{0} + {kA}_{ct} \qquad ( Eq . 5 )$$

Accordingly, one can consider that the capacitances of eAir devices according to example embodiments are linearly proportional to their liquid-electrode contact areas within the designed structures.

In one embodiment, a pressure sensor structure is provided comprising a chamber having a closed bottom and an open top; an array of working electrode surfaces disposed within the chamber and extending from the bottom of the chamber towards the open top; and a counter electrode formed outside the chamber; wherein the chamber with the working electrode surfaces disposed therein is configured to entrap air when the chamber is immersed in a liquid; wherein the working electrode surfaces are electrically contactable from outside the chamber; and wherein, when the air is entrapped in the chamber, changes in a capacitance between the working electrode and the counter electrode are a measure for pressure changes in the liquid in which the chamber is immersed.

The working electrode surfaces may be formed on inner walls of the chamber.

The working electrode surfaces may be formed on pillars formed on the bottom of the chamber. The pillars may comprise pointed tip portions such as dome-shaped or cone shaped tops, preferably with gradual tangential transitions between bases of the pointed tip portions and a body of the pillars.

The working electrode surfaces may be modified to provide a hydrophobic interface to the liquid. The working electrode surfaces may be modified to provide an advancing contact angle of the liquid under increasing pressure on the liquid that is greater than a static contact angle for water on a hydrophobic surface and less than 160°, preferably less than 135°, more preferably less than 115° and yet more preferably less than 112°.

The working electrode surfaces may be modified to provide a contact angle hysteresis between advancing contact angle of the liquid under increasing pressure on the liquid and receding contact angle of the liquid under decreasing pressure on the liquid of less than 10°, preferably less than 5°, and more preferably less than 3°.

The capacitance between the working electrode surfaces and the counter electrodes may comprise electric double layers at the working electrode surfaces to liquid interface and at the liquid to counter electrode surfaces.

The pressure sensor structure may comprise an encapsulation structure for encapsulating the chamber and the counter electrode with the chamber immersed in the liquid and the air entrapped in the chamber, such that the changes in the capacitance between the working electrode and the counter electrode are a measure for pressure changes on the encapsulation structure.

The chamber and the counter electrode may be formed on a rigid substrate.

The chamber and the counter electrode may be formed on a flexible substrate.

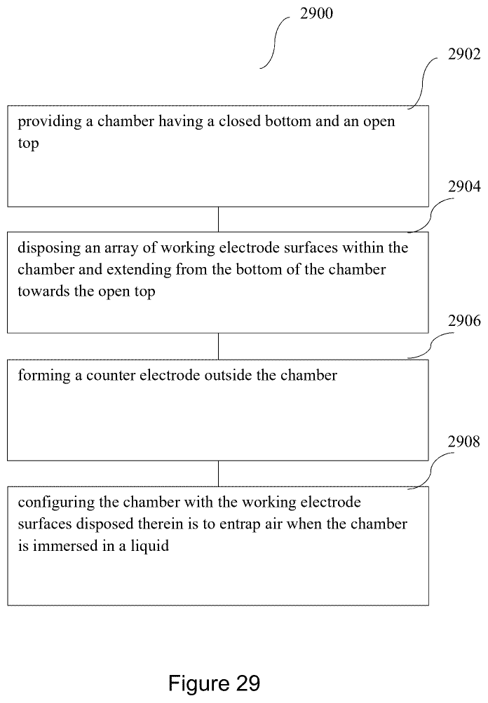

FIG. 29 shows a flowchart 2900 illustrating a method of fabricating a pressure sensor structure, according to an example embodiment. At step 2902, a chamber having a closed bottom and an open top is provided. At step 2904, an array of working electrode surfaces is disposed within the chamber and extending from the bottom of the chamber towards the open top. At step 2906, a counter electrode is formed outside the chamber. At step 2908, the chamber with the working electrode surfaces disposed therein is configured is to entrap air when the chamber is immersed in a liquid, wherein the working electrode surfaces are electrically contactable from outside the chamber, and wherein, when the air is entrapped in the chamber, changes in a capacitance between the working electrode and the counter electrode are a measure for pressure changes in the liquid in which the chamber is immersed.

The method may comprise forming the working electrode surfaces on inner walls of the chamber.

The method may comprise forming the working electrode on pillars formed on the bottom of the chamber. The method may comprise providing the pillars with pointed tip portions such as dome-shaped or cone shaped tops, preferably with gradual tangential transitions between bases of the pointed tip portions and a body of the pillars.

The method may comprise modifying the working electrode surfaces to provide a hydrophobic interface to the liquid. The method may comprise modifying the working electrode surfaces to provide an advancing contact angle of the liquid under increasing pressure on the liquid that is greater than a static contact angle for water on a hydrophobic surface and less than 160°, preferably less than 135°, more preferably less than 115° and yet more preferably less than 112°.

The method may comprise modifying the working electrode surfaces to provide a contact angle hysteresis between advancing contact angle of the liquid under increasing pressure on the liquid and receding contact angle of the liquid under decreasing pressure on the liquid of less than 10°, preferably less than 5°, and more preferably less than 3°.

The capacitance between the working electrode surfaces and the counter electrodes may comprise electric double layers at the working electrode surfaces to liquid interface and at the liquid to counter electrode surfaces.

The method may comprise encapsulating the chamber and the counter electrode with the chamber immersed in the liquid and the air entrapped in the chamber in an encapsulation structure, such that the changes in the capacitance between the working electrode and the counter electrode are a measure for pressure changes on the encapsulation structure.

The method may comprise forming the chamber and the counter electrode on a rigid substrate.

The method may comprise forming the chamber and the counter electrode on a flexible substrate.

In one embodiment, a method of using the pressure sensor structure of any one of the embodiments to measure pressure is provided, comprising the step of monitoring changes in the capacitance between the working electrode and the counter electrode as the measure for the pressure changes in the liquid in which the chamber is immersed.

The encapsulation structure may encapsulate the chamber and the counter electrode with the chamber immersed in the liquid and the air entrapped in the chamber, and the method may comprise monitoring the changes in the capacitance between the working electrode and the counter electrode as a measure for pressure changes on the encapsulation structure.

In one embodiment, a device comprising the pressure sensor structure of any one of the embodiments is provided.

The device may comprise a surgical grasper with one or more receptors formed on one claw thereof, wherein the receptors are in closed fluid communication with one or more of the pressure sensor structures such that changes in the capacitance between the working electrode and the counter electrode of the one or more pressure sensor structures are a measure for pressure changes on the one or more receptors.

The device may comprise a wireless module for transmitting data representing the changes in the capacitance between the working electrode and the counter electrode.

Embodiments of the present invention can have one or more of the following features and associated advantages/benefits:

| Feature | Benefit/Advantage |
|---|---|
| Using electrical double layer effect | High sensitivity, linear dependency |
| | between contact area and output |
| | capacitance |
| Using the aero-elasticity of | High sensitivity, predictable volume |
| entrapped air | compressibility |
| Using multiscale ultra-slippery | Ultralow hysteresis |
| surface | |
| Using Hexagon-walled pillar-array | Ultrahigh linearity |
| microstructures | |

### Industrial Applications of Example Embodiments

Conventional pressure sensors that are made of only solid-state materials suffer from a trilemma in simultaneously achieving high sensitivity, hysteresis, and linearity performance in liquid environments. Embodiments of the present invention addresses the trilemma by employing the aero-elasticity, a delicate structural design, a multiscale ultraslippery surface, and the electrical double layer effect at the electrode-liquid interface. The sensor according to a preferred embodiment can achieve the best pressure sensing performance to-date of highest linearity (R-square=0.99944±0.00015, non-linearity=(1.49±0.17)%) to-date, to the inventors” knowledge. The sensor simultaneously exhibited ultra-low hysteresis ((1.34±0.20)%) and high sensitivity (79.1±4.3 pF/kPa) in liquid environments. The sensor can work in complex liquid environments, such as in turbulence, in vivo, and can also sense acoustic vibrations.

Aspects of the systems and methods described herein, such as the generating of the sensor output, may be implemented on computing device(s), including cloud-based computing device(s) and/or Internet-of-Things computing device(s), for example as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the system include: microcontrollers with memory (such as electronically erasable programmable read only memory (EEPROM)), embedded microprocessors, firmware, software, etc. Furthermore, aspects of the system may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc. The various functions or processes disclosed herein may be described as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. When received into any of a variety of circuitry (e.g. a computer), such data and/or instruction may be processed by a processing entity (e.g., one or more processors).

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive. Also, the invention includes any combination of features described for different embodiments, including in the summary section, even if the feature or combination of features is not explicitly specified in the claims or the detailed description of the present embodiments.

In general, in the following claims, the terms used should not be construed to limit the systems and methods to the specific embodiments disclosed in the specification and the claims, but should be construed to include all processing systems that operate under the claims. Accordingly, the systems and methods are not limited by the disclosure, but instead the scope of the systems and methods is to be determined entirely by the claims.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

### REFERENCES

[1] Kang, S.-K. et al. Bioresorbable silicon electronic sensors for the brain. Nature 530, 71-76 (2016).
[2] Shin, J. et al. Bioresorbable pressure sensors protected with thermally grown silicon dioxide for the monitoring of chronic diseases and healing processes. Nat. Biomed. Eng. 3, 37-46 (2019).
[3] Ouyang, H. et al. A bioresorbable dynamic pressure sensor for cardiovascular postoperative care. Adv. Mater. 33, 2102302 (2021).
[4] Xu, R. et al. Superhydrophobic WS2-nanosheet-wrapped sponges for underwater detection of tiny vibration. Adv. Sci. 5, 1700655 (2018).
[5] Gao, Y. et al. Hydrogel microphones for stealthy underwater listening. Nat. Commun. 7, 12316 (2016).
[6] Tang, L. et al. Ultraminiature and flexible sensor based on interior corner flow for direct pressure sensing in biofluids. Small 15, 1900950 (2019).
[7] Starr, P., Bartels, K., Agrawal, C. M. & Bailey, S. A thin-film pressure transducer for implantable and intravascular blood pressure sensing. Sens. Actuators Phys. 248, 38-45 (2016).
[8] Luo, X. & Gianchandani, Y. B. A 100 μm diameter capacitive pressure sensor with 50 MPa dynamic range. J. Micromech. Microeng. 26, 045009 (2016).
[9] Hong, W. et al. An ultraminiature and flexible pressure sensor based on electrical-double-layer capacitance for catheter-tip applications. In 2018 IEEE Micro Electro Mechanical Systems (MEMS) 451-454 (IEEE, 2018).
[10] Shin, J. et al. Bioresorbable optical sensor systems for monitoring of intracranial pressure and temperature. Sci. Adv. 5, eaaw1899 (2019).
[11] Wong, T.-S. et al. Bioinspired self-repairing slippery surfaces with pressure-stable omniphobicity. Nature 477, 443-447 (2011).
[12] Wang, J. et al. Viscoelastic solid-repellent coatings for extreme water saving and global sanitation. Nat. Sustain. 2, 1097-1105 (2019).

1. A pressure sensor structure comprising:
a chamber having a closed bottom and an open top;
an array of working electrode surfaces disposed within the chamber and extending from the bottom of the chamber towards the open top; and
a counter electrode formed outside the chamber;
wherein the chamber with the working electrode surfaces disposed therein is configured to entrap air when the chamber is immersed in a liquid;
wherein the working electrode surfaces are electrically contactable from outside the chamber; and
wherein, when the air is entrapped in the chamber, changes in a capacitance between the working electrode and the counter electrode are a measure for pressure changes in the liquid in which the chamber is immersed.

2. The pressure sensor structure of claim 1, wherein the working electrode surfaces are formed on inner walls of the chamber.

3. The pressure sensor structure of claim 1, wherein the working electrode surfaces are formed on pillars formed on the bottom of the chamber, and optionally wherein the pillars comprise pointed tip portions such as dome-shaped or cone shaped tops, preferably with gradual tangential transitions between bases of the pointed tip portions and a body of the pillars.

4. (canceled)

5. The pressure sensor structure of claim 1, wherein the working electrode surfaces are modified to provide a hydrophobic interface to the liquid, and optionally wherein the working electrode surfaces are modified to provide an advancing contact angle of the liquid under increasing pressure on the liquid that is greater than a static contact angle for water on a hydrophobic surface and less than 160°, preferably less than 135°, more preferably less than 115° and yet more preferably less than 112°.

6. (canceled)

7. The pressure sensor structure of claim 5, wherein the working electrode surfaces are modified to provide a contact angle hysteresis between advancing contact angle of the liquid under increasing pressure on the liquid and receding contact angle of the liquid under decreasing pressure on the liquid of less than 10°, preferably less than 5°, and more preferably less than 3°.

8. The pressure sensor structure of claim 1, wherein the capacitance between the working electrode surfaces and the counter electrodes comprises electric double layers at the working electrode surfaces to liquid interface and at the liquid to counter electrode surfaces.

9. The pressure sensor structure of claim 1, comprising an encapsulation structure for encapsulating the chamber and the counter electrode with the chamber immersed in the liquid and the air entrapped in the chamber, such that the changes in the capacitance between the working electrode and the counter electrode are a measure for pressure changes on the encapsulation structure.

10. The pressure sensor structure of claim 1, wherein the chamber and the counter electrode are formed on a rigid substrate or wherein the chamber and the counter electrode are formed on a flexible substrate.

11. (canceled)

12. A method of fabricating a pressure sensor structure comprising the steps of:
providing a chamber having a closed bottom and an open top;
disposing an array of working electrode surfaces within the chamber and extending from the bottom of the chamber towards the open top;
forming a counter electrode outside the chamber; and
configuring the chamber with the working electrode surfaces disposed therein is to entrap air when the chamber is immersed in a liquid;
wherein the working electrode surfaces are electrically contactable from outside the chamber; and
wherein, when the air is entrapped in the chamber, changes in a capacitance between the working electrode and the counter electrode are a measure for pressure changes in the liquid in which the chamber is immersed.

13. The method of claim 12, comprising forming the working electrode surfaces on inner walls of the chamber.

14. The method of claim 12, comprising forming the working electrode on pillars formed on the bottom of the chamber, and optionally comprising providing the pillars with pointed tip portions such as dome-shaped or cone shaped tops, preferably with gradual tangential transitions between bases of the pointed tip portions and a body of the pillars.

15. (canceled)

16. The method of claim 12, comprising modifying the working electrode surfaces to provide a hydrophobic interface to the liquid, and optionally comprising modifying the working electrode surfaces to provide an advancing contact angle of the liquid under increasing pressure on the liquid that is greater than a static contact angle for water on a hydrophobic surface and less than 160°, preferably less than 135°, more preferably less than 115° and yet more preferably less than 112°.

17. (canceled)

18. The method of claim 16, comprising modifying the working electrode surfaces to provide a contact angle hysteresis between advancing contact angle of the liquid under increasing pressure on the liquid and receding contact angle of the liquid under decreasing pressure on the liquid of less than 10°, preferably less than 5°, and more preferably less than 3°.

19. The method of claim 12, wherein the capacitance between the working electrode surfaces and the counter electrodes comprises electric double layers at the working electrode surfaces to liquid interface and at the liquid to counter electrode surfaces.

20. The method of claim 12, comprising encapsulating the chamber and the counter electrode with the chamber immersed in the liquid and the air entrapped in the chamber in an encapsulation structure, such that the changes in the capacitance between the working electrode and the counter electrode are a measure for pressure changes on the encapsulation structure.

21. The method of claim 12, comprising forming the chamber and the counter electrode on a rigid substrate, or comprising forming the chamber and the counter electrode on a flexible substrate.

22. (canceled)

23. A method of using the pressure sensor structure of claim 1 to measure pressure, comprising the step of monitoring changes in the capacitance between the working electrode and the counter electrode as the measure for the pressure changes in the liquid in which the chamber is immersed.

24. The method of claim 23, wherein the encapsulation structure encapsulates the chamber and the counter electrode with the chamber immersed in the liquid and the air entrapped in the chamber, and the method comprises monitoring the changes in the capacitance between the working electrode and the counter electrode as a measure for pressure changes on the encapsulation structure.

25. A device comprising the pressure sensor structure of claim 1.

26. The device of claim 25, wherein the device comprises a surgical grasper with one or more receptors formed on one claw thereof, wherein the receptors are in closed fluid communication with one or more of the pressure sensor structures such that changes in the capacitance between the working electrode and the counter electrode of the one or more pressure sensor structures are a measure for pressure changes on the one or more receptors or comprising a wireless module for transmitting data representing the changes in the capacitance between the working electrode and the counter electrode.

27. (canceled)
